(19) United States
(12) Patent Application Publication
(10) Pub. No.: US 2026/0066122 A1
(43) Pub. Date: Mar. 5, 2026

(54) SYSTEMS AND METHODS FOR HIGH-THROUGHPUT PAN-CANCER GENETIC AND PHENOTYPIC BIOMARKER SCREENING

(71) Applicant: PAIGE.AI, Inc., New York, NY (US)

(72) Inventors: Yikan WANG, Vancouver (CA); Ludmila TRLIFAJ TYDLITATOVA, Vienna (AT); Jeremy Daniel KUNZ, New York, NY (US); Gerard OAKLEY, Fishers, IN (US); Ran GODRICH, New York, NY (US); Matthew LEE, London (GB); Razik YOUSFI, Brooklyn, NY (US); Thomas FUCHS, New York, NY (US); David S. KLIMSTRA, Middletown, NY (US); Siqi LIU, New York, NY (US)

(21) Appl. No.: 19/318,866

(22) Filed: Sep. 4, 2025

Related U.S. Application Data
(60) Provisional application No. 63/691,037, filed on Sep. 5, 2024.

(51) Int. Cl. G16H 50/20; G16H 30/40

(52) U.S. Cl. CPC G16H 50/20; G16H 30/40

(57) ABSTRACT

Disclosed are systems and methods for processing at least one digital medical image to predict a first biomarker, including receiving the at least one digital medical image of one or more tissues of a patient, the at least one digital medical image including a plurality of tiles, analyzing, via a foundation model, the plurality of tiles to determine an embedding vector for each of the plurality of tiles, the foundation model having been trained to predict embedding vectors at a tile-level based on a plurality of digital medical images, and analyzing, via an aggregator model, the embedding vector for each of the plurality of tiles to predict the first biomarker of the digital medical image, wherein the aggregator model includes an attention mechanism configured to aggregate the embedding vector for each of the plurality of tiles into at least one slide-level prediction.

### CROSS-REFERENCE TO RELATED APPLICATIONS

This application claims priority to U.S. Provisional Application No. 63/691,037, filed on Sep. 5, 2024, the entire disclosure of which is hereby incorporated by reference in its entirety.

This application further incorporates by reference U.S. Non-Provisional application Ser. No. 18/521,903, filed on Nov. 28, 2023, the entire disclosure of which is hereby incorporated herein by reference in its entirety.

### TECHNICAL FIELD

Various embodiments of the present disclosure relate generally to large-scale image processing. More specifically, particular embodiments of the present disclosure relate to systems and methods for high-throughput pan-cancer genetic and phenotypic biomarker screening.

### BACKGROUND

Many molecular alterations serve as clinically prognostic or therapy-predictive biomarkers, typically detected using single or multi-gene molecular assays. However, these assays are expensive, tissue destructive and often take weeks to complete. Further, conventional analysis of these molecular alterations often require training individual models for each biomarker or cancer type, which requires extremely large data sets. Conventional techniques, including the foregoing, fail to account for the need to analyze large quantities of data, often across various modalities. Systems and/or methods that operate in a pan-cancer and/or pan-tissue manner are needed.

The foregoing general description and the following detailed description are exemplary and explanatory only and are not restrictive of the disclosure. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the present disclosure, systems and methods are disclosed for high-throughput pan-cancer genetic and phenotypic biomarker screening.

In one aspect, a computer-implemented method for processing at least one digital medical image to predict a first biomarker is disclosed. The method may include receiving the at least one digital medical image of one or more tissues of a patient, the at least one digital medical image including a plurality of tiles, analyzing, via a foundation model, the plurality of tiles to determine an embedding vector for each of the plurality of tiles, the foundation model having been trained to predict embedding vectors at a tile-level based on a plurality of digital medical images, and analyzing, via an aggregator model, the embedding vector for each of the plurality of tiles to predict the first biomarker of the digital medical image, wherein the aggregator model includes an attention mechanism configured to aggregate the embedding vector for each of the plurality of tiles into at least one slide-level prediction.

In another aspect, a method for training an aggregator model to predict at least one biomarker is disclosed. The method may include receiving a plurality of digital medical images associated with a plurality of patients, receiving genomic abnormality data associated with the plurality of patients, and training the aggregator model to predict the at least one biomarker based on the plurality of digital medical images and the genomic abnormality data.

In a further aspect, a system for processing at least one digital medical image to predict a first biomarker is disclosed. The system may include at least one memory storing instructions, and at least one processor configured to execute the instructions to perform operations. The operations may include receiving the at least one digital medical image of one or more tissues of a patient, the at least one digital medical image including a plurality of tiles, analyzing, via a foundation model, the plurality of tiles to determine an embedding vector for each of the plurality of tiles, and analyzing, via an aggregator model, the embedding vector for each of the plurality of tiles to predict the first biomarker of the digital medical image. The foundation model may have been trained to predict embedding vectors at a tile-level based on a plurality of digital medical images. The aggregator model may include an attention mechanism configured to aggregate the embedding vector for each of the plurality of tiles into at least one slide-level prediction.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A depicts a block diagram of an exemplary system for predicting biomarkers, according to one or more embodiments.

FIG. 1B depicts a block diagram of an exemplary system for foreground detection, according to one or more embodiments.

FIG. 1C depicts a block diagram of an exemplary system for vector generation, according to one or more embodiments.

FIG. 1D depicts a block diagram of an exemplary system for biomarker prediction, according to one or more embodiments.

FIG. 1E depicts a block diagram of an exemplary system for tumor analysis, according to one or more embodiments.

FIG. 2 depicts a schematic of an exemplary system for predicting biomarkers, according to one or more embodiments.

FIG. 3 depicts a schematic of exemplary heatmaps, according to one or more embodiments.

FIG. 4 depicts a flow diagram for an exemplary process for predicting biomarkers, according to one or more techniques.

FIGS. 5A-5F depict flow diagrams for exemplary training methods, according to one or more techniques.

FIG. 6 depicts a schematic for an exemplary process for training a machine learning model for predicting biomarkers, according to one or more techniques.

FIG. 7 depicts an example system or device that may execute techniques presented herein, according to one or more techniques.

Notably, for simplicity and clarity of illustration, certain aspects of the figures depict the general configuration of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments.

### DETAILED DESCRIPTION OF EMBODIMENTS

Various embodiments of the present disclosure relate generally to large-scale image processing. More specifically, particular embodiments of the present disclosure relate to systems and methods for high-throughput pan-cancer genetic and phenotypic biomarker screening.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

Any suitable system infrastructure may be put into place to allow user control of an interactive audiovisual environment, and engagement assessment. The following discussion provide a brief, general description of a suitable computing environment in which the present disclosure may be implemented. In one embodiment, any of the disclosed systems, methods, and/or graphical user interfaces may be executed by or implemented by a computing system. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Techniques disclosed herein may describe systems and related methods for large-scale image processing using foundation models. Foundation models may include large-scale deep neural networks trained in a self-supervised manner and adaptable for downstream tasks. For example, millions of slides across hundreds of tissue types may be analyzed by a foundation model for universal whole slide representation for pattern discovery applied to cancer detection or segmentation, as well as one or more downstream prognostic clinical, or biomarker tasks.

In an exemplary use case, a computer-implemented method for processing a digital medical image to predict biomarkers of interest from the image may be described herein. The method may include the following steps.

The method may include receiving a digital medical image of one or more tissues of a patient. The digital medical image may include at least one of whole slide image (WSI), hematoxylin and eosin (H & E) stains, immunohistochemistry (IHC) slides, immunofluorescent slides, or CT scans.

The method may include splitting the digital medical image into a set of tiles.

The method may include converting the set of tiles into an embedding vector. Converting the set of tiles into an embedding vector may further include: inserting the set of tiles into a foreground detection model, the foreground detection model being a fully convolutional neural network; and determining a set of foreground tiles to further analyze. Converting the set of tiles into an embedding vector may include determining the set of tiles by inserting the set of tiles into a Virchow2 model.

The method may include determining a set of foreground tiles to further analyze.

The method may include inputting the embedding vector into a feed-forward network, wherein the feed-forward network includes an attention mechanism configured to aggregate tile-level embeddings into one or more slide level predictions. The feed-forward network may be configured to generate genomic biomarkers, wherein the genomic biomarkers represent genomic abnormalities in one or more cancer types

The method may include determining, by the feed-forward network, a biomarker of interest for the digital medical image. The biomarker of interest may be one of a genetic alteration biomarkers, a historic-subtype biomarkers, a treatment-associated biomarker, or a pathway & chromosomal instability biomarker.

The method may further include suggesting, based on the determined biomarker of interest, a pre-screening for the patient; or determining, based on the biomarker of interest, a suggested treatment for the patient; and/or determining, based on the biomarker of interest, a diagnosis of a specific historic subtype of cancer.

The method may further include determining, by the feed-forward network, a second biomarker of interest for the digital medical image, the second biomarker of interest being a separate type of biomarker interest compared to the biomarker of interest.

One or more embodiments described herein may be configured to predict a wide range of molecular biomarkers across different cancer types. The system may be configured to analyze all tissue types to discover how multiple tests can be combined to more effectively predict disease, outcome, and/or treatment response.

Advantageously, the unified model described herein may simultaneously predict a wide range of clinically relevant molecular biomarkers across cancer type. The described system may offer potential to guide therapy selection, improve treatment efficacy, accelerate patient screening for clinical trials and provoke the interrogation of new therapeutic. The model described herein may significantly enhances the efficiency of biomarker screening across various cancer types, identifying not only clinically relevant genomic abnormalities but also histology's characterized by specific genomic alterations.

### SUMMARY

Many molecular alterations may serve as clinically prognostic or therapy predictive biomarkers, typically detected using single or multi-gene molecular assays. However, these assays may be expensive, tissue destructive and often take weeks to complete. Using artificial intelligence (AI) on routine H & E WSIs may offer a fast and economical approach to screen for multiple molecular biomarkers. The system described herein may include a high-throughput AI-based system leveraging Virchow2, a foundation model pre-trained on 3 million slides, to interrogate genomic features previously determined by a next-generation sequencing (NGS) assay, using a large sample size of scanned hematoxylin and eosin (H & E) whole slide images (WSIs) from a large sample size of cancer patients. Unlike traditional methods that train individual models for each biomarker or cancer type, the system described herein may employ a unified model to simultaneously predict a wide range of clinically relevant molecular biomarkers across cancer types. By training the network to replicate a targeted biomarker panel of, for example, 505 genes, it may have identified eighty high performing biomarkers with a mean Area Under the Receiver Operating Characteristic (AUROC) of 0.89 in fifteen most common cancer types. In addition, forty biomarkers may have demonstrated strong associations with specific cancer histologic subtypes. Furthermore, fifty-eight biomarkers may have been associated with targets frequently assayed clinically for therapy selection and response prediction. The model may also predict the activity of five canonical signaling pathways, identify defects in DNA repair mechanisms, and predict genomic instability measured by tumor mutation burden, microsatellite instability (MSI), and chromosomal instability (CIN). The proposed model can offer potential to guide therapy selection, improve treatment efficacy, accelerate patient screening for clinical trials and provoke the interrogation of new therapeutic targets.

### INTRODUCTION

Modern cancer treatment decisions may rely on several important factors such as the patient age, life expectancy, and the specific type, grade, and stage of their cancer. Tools such as nomograms, which can inform clinical treatment guidelines, may help doctors estimate patient prognosis and recommend the best treatment options. Treatments can range from monitoring the cancer without active treatment to surgery, hormone therapy, chemotherapy, radiotherapy, targeted therapy, immunotherapy and combinations thereof.

In recent years, there may have been a significant push towards developing personalized treatments for patients based on the genetic alterations within their cancers. This may have led to rapid advances in molecular biomarker tests, including single and multi-gene assays, which may analyze tissue, blood, and body fluid samples to identify targetable genomic alterations and guide doctors in making more informed treatment decisions. Based on improved analysis of cancer genomics, novel targets with potential clinical relevance are reported every year. For example, genomic alterations such as androgen receptor (AR) variants may help predict endocrine versus chemotherapy resistance in metastatic castrate resistant prostate cancer (mCRPC), or BRCA1/2 germline mutations predict poly(ADP-ribose) polymerase (PARP) inhibitor response in the treatment of high-risk early stage HER2-negative breast cancer.

Commercially available multi-gene tests in localized disease, such as Oncotype DX Breast Recurrence Score Test, MammaPrint test, Oncotype DX Genomics Prostate Score, ProMark, Decipher and Prolaris, may provide significant prognostic guidance across specific cancers, complementing routine clinicopathological factors in clinical decision-making. However, these tests may often be costly, time-consuming, and require substantial tissue samples, posing challenges particularly in small core biopsies or those with limited tumor cells. To address these limitations, newer assays using minimally invasive approaches, such as “liquid biopsy” blood draws for circulating tumor cell or circulating nucleic acid analysis, have been developed. Yet, the systems may also face challenges with sample quantity, standardization of cell collection and stabilization procedures, and suffer from sensitivity and specificity issues due to non-tumor mutated clones present even in the blood of patients without cancer. Thus, there may be a growing need for digital biomarkers derived from widely available digital H & E whole slide images to rapidly and cost-effectively screen patient samples for multiple genomic biomarkers in a robust and tissue sparing manner. This approach may enable the swift identification of cases that require definitive genomic testing for clinical management and appropriate therapy selection while excluding cases where such testing is unlikely to be fruitful, thereby improving turnaround time and reducing testing costs without compromising clinical care.

Beyond their clinical impact, digital biomarkers may offer substantial advantages for the pharmaceutical industry, particularly for drug development and clinical trials. Digital biomarkers may facilitate novel target identification and drug discovery, leading to the development of more effective targeted therapies. They may enhance patient stratification, which may improve the cost efficiency and success rates of clinical trials. Additionally, digital biomarkers may support the creation of companion and complementary diagnostics to help personalize therapy selection to the genetic profile of the patient's tumor. By optimizing resource use and accelerating decision-making processes, digital biomarkers may contribute to cost savings in drug discovery and improve access to novel targeted therapies by reducing the cost of necessary clinical screening for healthcare systems and payers.

Systems may demonstrate exemplary methods to identify morphological features associated with genomic abnormalities in routine H & E histopathology images across various cancer types, enabling the prediction of digital molecular biomarkers. However, previous systems may have focused on only one biomarker for a specific tissue or cancer type at a time, a method that is notably inefficient. This inefficiency may stem from two issues: (1) each biomarker suffers from limited training data, hindering generalization, even though they may share morphological phenotypes, and (2) the costs associated with developing individual models are significant. Therefore, the feasibility of detecting many digital biomarkers simultaneously may not have been previously reported.

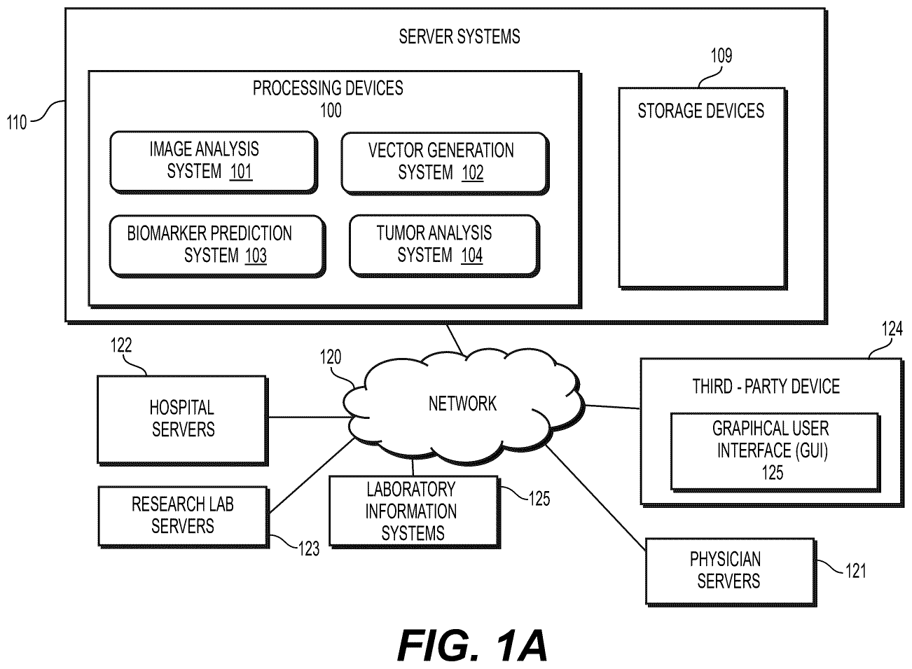

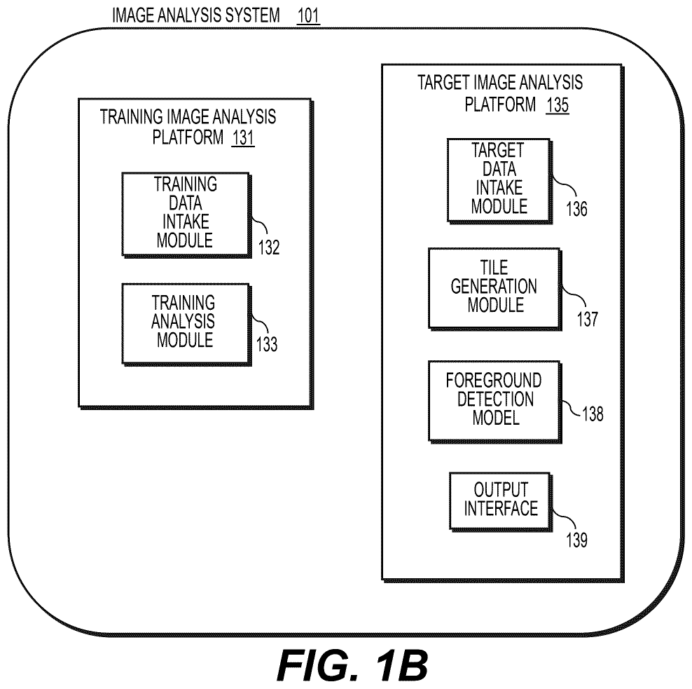

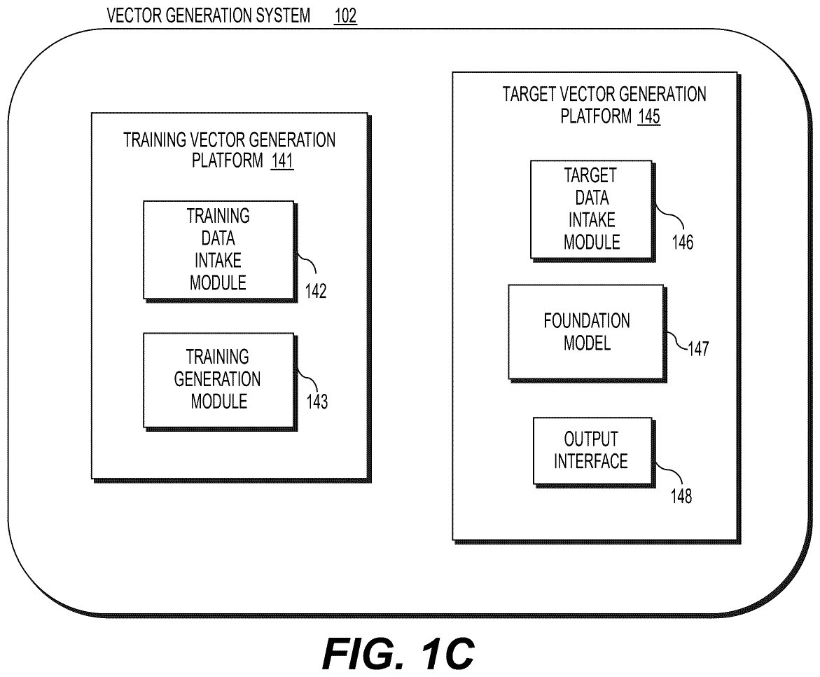

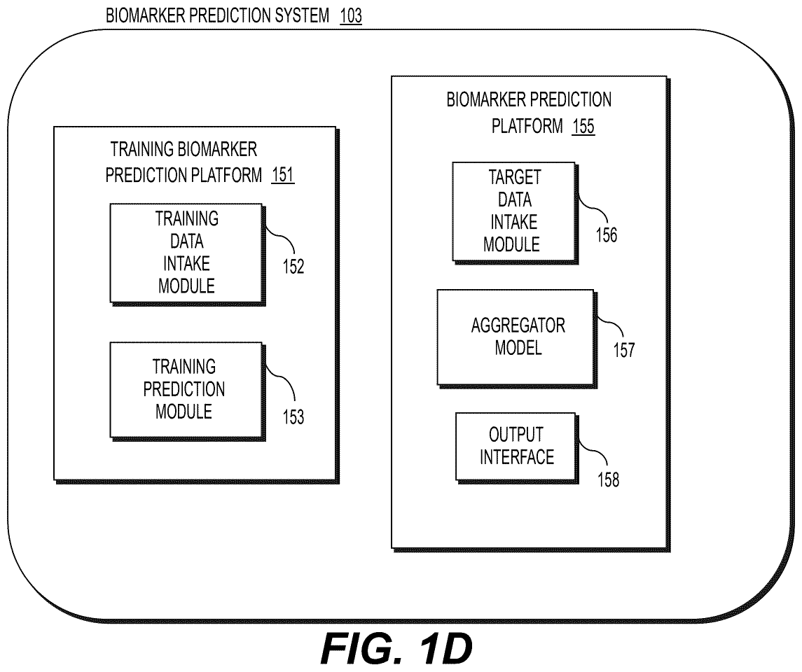

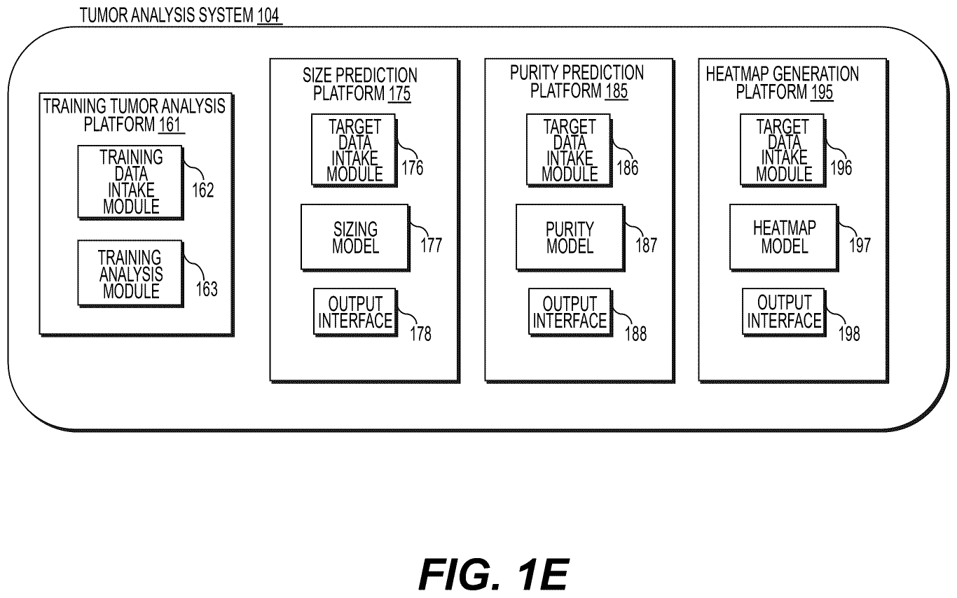

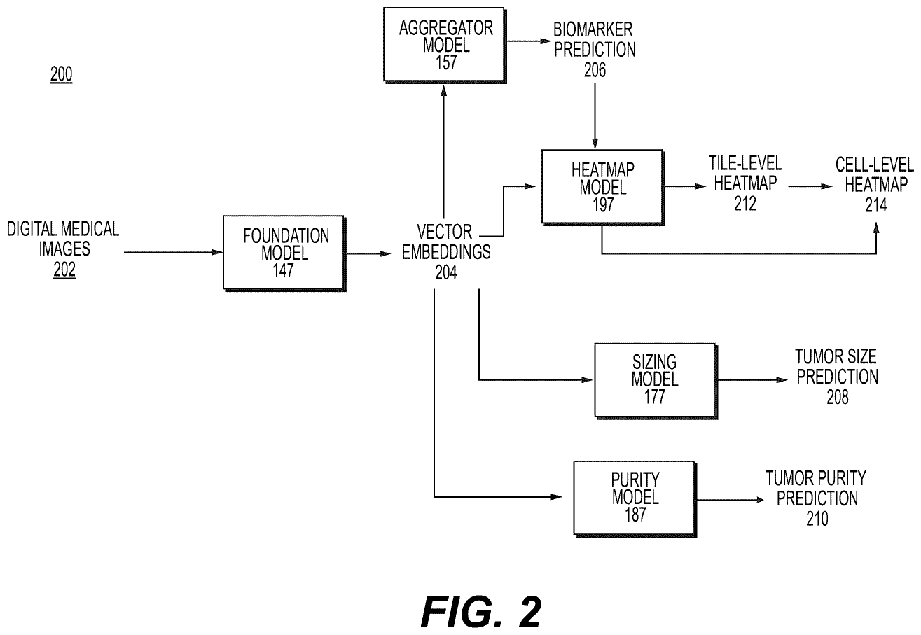

A universal model may be essential to identify all clinically relevant molecular biomarkers from tissue-agnostic H & E whole slide images for all cancer types, which would benefit both clinical applications and pharmaceutical research. The systems and methods described herein may, we define an approach to high-throughput screening for genomic abnormalities applicable to all cancer types using routine H & E whole slide images. An exemplary system that may perform this is depicted in FIG. 1 below. By leveraging image representations from a foundation model pre-trained on millions of slides, the system described herein may be modeled to simultaneously predict 1,228 genomic biomarkers, representing genomic abnormalities in 70 human cancers (as depicted in FIG. 2 below), by training and testing on a cohort of exemplary whole slide images (WSIs). Patients in the cohort may have had associated known ground truth genomic abnormalities from the paired tumor-normal targeted sequencing using the Food and Drug Administration (FDA)-cleared Integrated Mutation Profiling of Actionable Targets (IMPACT) assay. Additional genomic features such as deficient mismatch repair (dMMR) status may have been confirmed by immunohistochemistry (IHC) assay. The model described herein may have identified at least 391 genomic alteration biomarkers with AUC>0.75 in the fifteen most common cancer types treated. Evaluating phenotype-genotype associations may have revealed at least 40 histologic biomarkers, while 58 treatment-associated biomarkers were identified as predictors of response to FDA-approved drugs. The genomic biomarkers may have been further validated using diagnostic slides and genomic data (e.g., the TCGA Pan-Cancer Atlas Cohort). The model described herein may significantly enhance the efficiency of biomarker screening across various cancer types, identifying not only clinically relevant genomic abnormalities but also histologies characterized by specific genomic alterations. The system may be utilized in patient screening for definitive genome analysis, guiding treatment selection, and identifying new therapeutic targets.

FIGS. 1A-1E depict block diagrams of an exemplary system for predicting biomarkers, according to one or more embodiments. Illustrated in FIG. 1A is an electronic network 120 that may be connected to physician servers 121, hospital servers 122, clinical trial servers 123, research lab servers 124, and/or laboratory information systems 125, for example, through one or more computers, servers, and/or handheld mobile devices. According to an exemplary aspect of the present disclosure, network 120 may be connected to server systems 110, which may include one or more processing devices 100, e.g., configured to run or execute an image analysis system 101, a vector generation system 102, a biomarker prediction system 103, a tumor analysis system 104, and/or storage devices 109. Image analysis system 101 may be configured for foreground detection. Vector generation system 102 may be configured for vector generation. Biomarker prediction system 103 may be configured for biomarker prediction. Tumor analysis system 104 may be configured for tumor analysis. While image analysis system 101, vector generation system 102, biomarker prediction system 103, and tumor analysis system 104 are depicted as separate systems in FIG. 1A, it should be understood that, in other examples, these systems may be sub-systems of a larger system.

Physician servers 121, hospital servers 122, clinical trial servers 123, research lab servers 124, and/or laboratory information systems 125 may create or otherwise obtain data, such as digital medical images, biomarker or biomarker data, genomic variants (e.g., genetic abnormality data), histological subtype data, treatment association data, genomic pathway data, tumor mutation burden (TMB), microsatellite instability (MSI), or chromosomal instability data (e.g., genome instability index (GI), fraction of genome altered (FGA), tetraploidy, whole genome doubling (WGD), and/or loss-of-heterozygosity (LOH)), and/or clinical data. For example, the digital medical images may include digital pathology images, including one or more patients' whole slide image(s), cytology specimen(s), histopathology specimen(s), slide(s) of the cytology specimen(s), digitized images of the slide(s) of the histopathology specimen(s), or any combination thereof, that may be created or obtained. Additionally or alternatively, the digital medical images may include images of other modality types, including digital multiplex immunofluorescent images, digital multiplex immunohistochemistry images, magnetic resonance imaging (MRI), computed tomography (CT), X-ray, nuclear medicine imaging, or ultrasound, that may be created or obtained.

The biomarker or biomarker data may include at least one of a genetic alteration biomarker, a histologic-subtype biomarker, a treatment-associated biomarker, a pathway biomarker, a chromosomal instability biomarker, a transcriptomic biomarker, a proteomic biomarker, an epigenetic biomarker, a prognostic biomarker, etc. Genomic variants (e.g., genetic abnormality data) may include the genomic variation(s) that may give rise to a given phenotype (e.g., cancer phenotype). Histological subtype data may include the histological subtype associated with at least one digital medical image. Treatment association data may include data regarding treatment outcomes for a given treatment and particular disease type (e.g., cancer type), genotype (e.g., cancer genotype), phenotype (e.g., cancer phenotype), etc. Genomic pathway data may include data related to the genomic pathway of a disease. For example, genomic pathway data for a given cancer type may include the upstream and downstream physiological effects related to the cancer type, the cancer genotype, etc. Chromosomal instability data may include data relating to the stability of a chromosome, e.g., whether a chromosome is considered fragile.

Digital medical images, biomarker or biomarker data, genomic variants, histological subtype data, treatment association data, genomic pathway data, chromosomal instability data, clinical data, and/or other data may be communicated between server systems 110 and physician servers 121, hospital servers 122, clinical trial servers 123, research lab servers 124, and/or laboratory information systems 125 over network 120 in a digital or electronic format.

Server systems 110 may include one or more storage devices 109 for storing data, e.g., digital medical images, biomarker or biomarker data, genomic variants, histological subtype data, treatment association data, genomic pathway data, chromosomal instability data, clinical data, and/or other data, received from at least one of physician servers 121, hospital servers 122, clinical trial servers 123, research lab servers 124, and/or laboratory information systems 125. For example, vectors generated by vector generation system 102 may be stored within the one or more data stores, e.g., storage devices 109.

Server systems 110 may include processing devices 100 for processing the digital medical images and/or other above-described data stored in storage devices 109. In one aspect, at least one system of server systems 110 may be configured to generate a request for review based on at least one of: at least one digital medical image, foreground tile, at least one embedding vector, at least one biomarker prediction, and/or at least one heatmap. The at least one system of server systems 110 may be configured to transmit the request for review to a third-party device 124 (e.g., to be displayed via a graphical user interface 125 of third-party device 124).

Server systems 110 may include one or more machine learning tool(s) or capabilities. For example, processing devices 100 may execute one or more machine learning systems utilized by image analysis system 101, vector generation system 102, biomarker prediction system 103, and/or tumor analysis system 104. In some examples, outputs of the machine learning systems may be stored in storage devices 109 for use by other systems or processes, as described in detail below. Alternatively or in addition, the present disclosure (or portions of the system and methods of the present disclosure) may be performed on a local processing device (e.g., a laptop).

According to an exemplary aspect of the present disclosure, image analysis system 101 of FIG. 1B may be configured for foreground detection. The image analysis system 101 may include a training image analysis platform 131 and/or a target image analysis platform 135. The training image analysis platform 131, according to one technique, may create or receive one or more datasets of training data used to generate and train one or more machine learning models that, when implemented, detect foreground in the at least one digital medical image. According to one technique, the training image analysis platform 131 may include a plurality of software modules, including a training data intake module 132 and/or a training analysis module 133. The data and/or machine learning systems output by training image analysis platform 131 may be stored, e.g., in storage device 109, or used by other systems, e.g., target image analysis platform 135.

Training data intake module 132, according to one aspect, may create or receive training data (e.g., digital medical images, foreground data, background data, etc.) that may be used to train one or more machine learning systems for foreground detection. The training data may be received from any one or any combination of server systems 110, physician servers 121, hospital servers 122, clinical trial servers 123, research lab servers 124, and/or laboratory information systems 125. Training data may come from real sources (e.g., humans, animals, etc.) or may come from synthetic sources (e.g., graphics simulators, graphics rendering engines, 3D models, etc.).

The training analysis module 133 may generate, using the training data as input, one or more machine learning systems capable of foreground detection of the at least one digital medical image. In some examples, a third party may generate the one or more trained machine learning systems and provide the trained machine learning system(s) to server systems 110 for storage (e.g., in storage devices 109) and/or execution by image analysis system 101. Training analysis module 133 may train a graph neural network, a convolutional neural network, a transformer neural network, or any other suitable type of machine learning system for foreground detection. Training analysis module 133 may store the at least one digital medical image and detected foreground in a database, e.g., storage devices 109. Methods for training the one or more machine learning systems of training analysis module 133 are described herein.

According to one technique, the target image analysis platform 135 may include software modules, such as a target data intake module 136, a tile generation module 137, a foreground detection module 138, and an output interface 139. Target image analysis platform 135, according to one aspect, may receive a request for foreground detection for at least one digital medical image and execute one or more of the machine learning systems trained by training image analysis platform 131 for foreground generation. For example, the request may be received from any one or any combination of the physician servers 121, hospital servers 122, clinical trial servers 123, research lab servers 124, and/or laboratory information systems 125.

Target data intake module 136, according to one aspect, may create or receive target data (e.g., images, optionally clinical data, etc.) that may be used as an input for one or more trained machine learning systems for foreground detection. For example, target data intake module 136 may receive digital medical images, which may be used as an input for one or more trained machine learning systems. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). The target data intake module 136 may create or receive the one or more datasets of target data, e.g., digital medical images. For example, the datasets may include one or more datasets corresponding to digital medical images and/or, optionally, one or more datasets corresponding to clinical data. In some examples, a subset of target data may overlap between or among the various datasets for images and/or clinical data. The target datasets may be stored on a digital storage device, e.g., one of storages devices 109.

Tile generation module 137, according to one aspect, may generate a plurality of tiles for each of the at least one digital medical images that may be used as an input for one or more trained machine learning systems for foreground detection, e.g., foreground detection model 138. The plurality of tiles and/or the associated digital medical images may be stored on a digital storage device, e.g., one of storages devices 109.

Foreground detection model 138, according to one aspect, may be configured to analyze the plurality of tiles, e.g., to select a plurality of foreground tiles. The foreground detection model 138 may include any suitable machine learning systems, including but not limited to, graph neural networks, convolutional neural networks, transformer neural networks, etc. Foreground detection model 138 may execute the various machine learning systems generated by training image analysis platform 131, e.g., training analysis module 133, to facilitate the analysis of the plurality of tiles.

The output interface 139 may be used to output the plurality of tiles (e.g., to a screen, monitor, storage device, web browser, etc.). According to some techniques, output interface 139 may output the plurality of tiles for use as input in a subsequent process described herein. The plurality of tiles and other data produced or used by image analysis system 101 may be stored in one or storage devices 109.

According to an exemplary aspect of the present disclosure, vector generation system 102 of FIG. 1C may be configured for vector generation. The vector generation system 102 may include a training vector generation platform 141 and/or a target vector generation platform 145. The training vector generation platform 141, according to one technique, may create or receive one or more datasets of training data used to generate and train one or more machine learning models that, when implemented, generate vector(s) based on the at least one digital medical image, the plurality of tiles, the plurality of foreground tiles, etc. According to one technique, the training vector generation platform 141 may include a plurality of software modules, including a training data intake module 142 and/or a training generation module 143. The data and/or machine learning systems output by training vector generation platform 141 may be stored, e.g., in storage device 109, or used by other systems, e.g., target vector generation platform 145.

Training data intake module 142, according to one aspect, may create or receive training data (e.g., digital medical images, foreground data, background data, tiles, foreground tiles, etc.) that may be used to train one or more machine learning systems for vector generation. The training data may be received from any one or any combination of server systems 110, physician servers 121, hospital servers 122, clinical trial servers 123, research lab servers 124, and/or laboratory information systems 125. Training data may come from real sources (e.g., humans, animals, etc.) or may come from synthetic sources (e.g., graphics simulators, graphics rendering engines, 3D models, etc.).

The training generation module 143 may generate, using the training data as input, one or more machine learning systems capable of vector generation of the foreground tile(s). In some examples, a third party may generate the one or more trained machine learning systems and provide the trained machine learning system(s) to server systems 110 for storage (e.g., in storage devices 109) and/or execution by vector generation system 102. Training generation module 143 may train a graph neural network, a convolutional neural network, a transformer neural network, or any other suitable type of machine learning system for vector generation. Training generation module 143 may store the embedding vector(s) in a database, e.g., storage devices 109. Methods for training the one or more machine learning systems of training generation module 143 are described herein.

According to one technique, the target vector generation platform 145 may include software modules, such as a target data intake module 146, a foundation model 147, and an output interface 148. Target vector generation platform 145, according to one aspect, may receive a request for vector generation for at least one digital medical image and/or a plurality of tiles and execute one or more of the machine learning systems trained by training vector generation platform 141 for vector generation. For example, the request may be received from any one or any combination of the physician servers 121, hospital servers 122, clinical trial servers 123, research lab servers 124, and/or laboratory information systems 125.

Target data intake module 146, according to one aspect, may create or receive target data (e.g., tiles, images, optionally clinical data, etc.) that may be used as an input for one or more trained machine learning systems for vector generation. For example, target data intake module 146 may receive a plurality of tiles associated with digital medical images, which may be used as an input for one or more trained machine learning systems. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). The target data intake module 146 may create or receive the one or more datasets of target data, e.g., the plurality of tiles. For example, the datasets may include one or more datasets corresponding to a plurality of tiles and/or, optionally, digital medical images associated with the plurality of tiles. In some examples, a subset of target data may overlap between or among the various datasets for images and/or clinical data. The target datasets may be stored on a digital storage device, e.g., one of storages devices 109.

Foundation model 147, according to one aspect, may be configured to analyze the plurality of foreground tiles, e.g., to generate embedding vector(s) for each of the plurality of foreground tiles. In one aspect, foundation model 147 may be trained to predict embedding vectors at a tile-level based on the plurality of digital medical images. The foundation model 147 may include any suitable machine learning systems, including but not limited to, graph neural networks, convolutional neural networks, transformer neural networks, etc. For example, foundation model 147 may be a Virchow2 model. Foreground detection model 138 may execute the various machine learning systems generated by training image analysis platform 131, e.g., training analysis module 133, to facilitate the analysis of the plurality of tiles.

The output interface 148 may be used to output the embedding vector(s) (e.g., to a screen, monitor, storage device, web browser, etc.). According to some techniques, output interface 148 may output the embedding vector(s) for use as input in a subsequent process described herein. The embedding vector(s) and other data produced or used by vector generation system 102 may be stored in one or storage devices 109.

According to an exemplary aspect of the present disclosure, biomarker prediction system 103 of FIG. 1D may be configured for biomarker prediction. The biomarker prediction system 103 may include a training biomarker prediction platform 151 and/or a biomarker prediction platform 155. The training biomarker prediction platform 151, according to one technique, may create or receive one or more datasets of training data used to generate and train one or more machine learning models that, when implemented, predict biomarkers in the plurality of foreground tiles. According to one technique, the training biomarker prediction platform 151 may include a plurality of software modules, including a training data intake module 152 and/or a training prediction module 153. The data and/or machine learning systems output by training biomarker prediction platform 151 may be stored, e.g., in storage device 109, or used by other systems, e.g., biomarker prediction platform 155.

Training data intake module 152, according to one aspect, may create or receive training data (e.g., embedding vector(s), the plurality of tiles digital medical images, foreground data, background data, etc.) that may be used to train one or more machine learning systems for biomarker prediction. The training data may be received from any one or any combination of server systems 110, physician servers 121, hospital servers 122, clinical trial servers 123, research lab servers 124, and/or laboratory information systems 125. Training data may come from real sources (e.g., humans, animals, etc.) or may come from synthetic sources (e.g., graphics simulators, graphics rendering engines, 3D models, etc.).

The training prediction module 153 may generate, using the training data as input, one or more machine learning systems capable of biomarker prediction. In some examples, a third party may generate the one or more trained machine learning systems and provide the trained machine learning system(s) to server systems 110 for storage (e.g., in storage devices 109) and/or execution by biomarker prediction system 103. Training prediction module 153 may train a graph neural network, a convolutional neural network, a transformer neural network, or any other suitable type of machine learning system for biomarker prediction. Training prediction module 153 may store the biomarker prediction(s) in a database, e.g., storage devices 109. Methods for training the one or more machine learning systems of training prediction module 153 are described herein.

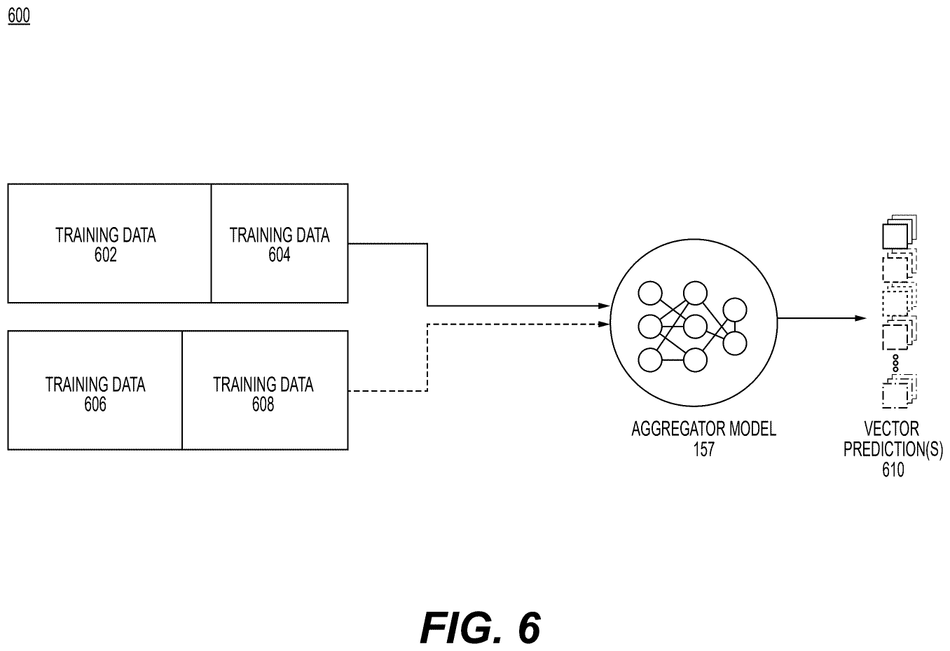

FIG. 6 depicts a flow diagram 600 for an exemplary process for training aggregator model 157 for predicting biomarkers, e.g., vector prediction(s) 610, according to one or more techniques. Training data 602 may include a training set of data, training data 604 may include a tuning set of data, training data 606 may include a first test set of data, and training data 608 may include a second test set of data. The aggregator model 157 may be configured to receive the tile embeddings of training data 602 and/or run validation during training on the training data 604. Once trained, the aggregator model 157 may be evaluated on at least one unseen test set

Returning to FIG. 1D, according to one technique, the biomarker prediction platform 155 may include software modules, such as a target data intake module 156, an aggregator 157, and an output interface 158. Biomarker prediction platform 155, according to one aspect, may receive a request for biomarker prediction for at least one digital medical image and execute one or more of the machine learning systems trained by training biomarker prediction platform 151 for biomarker prediction. For example, the request may be received from any one or any combination of the physician servers 121, hospital servers 122, clinical trial servers 123, research lab servers 124, and/or laboratory information systems 125.

Target data intake module 156, according to one aspect, may create or receive target data (e.g., embedding vector(s), plurality of tiles, images, optionally clinical data, etc.) that may be used as an input for one or more trained machine learning systems for biomarker prediction. For example, target data intake module 156 may receive embedding vector(s), which may be used as an input for one or more trained machine learning systems. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). The target data intake module 156 may create or receive the one or more datasets of target data, e.g., embedding vector(s). For example, the datasets may include one or more datasets corresponding to embedding vector(s) and/or, optionally, one or more datasets corresponding to digital medical images and/or clinical data. In some examples, a subset of target data may overlap between or among the various datasets for images and/or clinical data. The target datasets may be stored on a digital storage device, e.g., one of storages devices 109.

Aggregator model 157, according to one aspect, may be configured to analyze the embedding vector(s), e.g., to predict at least one biomarker. The aggregator model 157 may include an attention mechanism configured to aggregate the embedding vector for each of the plurality of tiles into at least one slide-level prediction. The aggregator model 157 may include any suitable machine learning systems, including but not limited to, graph neural networks, convolutional neural networks, transformer neural networks, etc. Aggregator model 157 may execute the various machine learning systems generated by training biomarker prediction platform 151, e.g., training prediction module 153, to facilitate the biomarker prediction. Aggregator model 157 may be configured to determine at least one biomarker (e.g., a first biomarker, a second biomarker, etc.). In some aspects, each biomarker may be a different biomarker type. For example, the second biomarker may be a different biomarker type than the first biomarker.

The output interface 158 may be used to output the biomarker prediction(s) (e.g., to a screen, monitor, storage device, web browser, etc.). According to some techniques, output interface 158 may output the biomarker prediction(s) for use as input in a subsequent process described herein. The biomarker prediction(s) and other data produced or used by biomarker prediction system 103 may be stored in one or storage devices 109.

According to an exemplary aspect of the present disclosure, tumor analysis system 104 of FIG. 1E may be configured for tumor analysis. The tumor analysis system 104 may include a training tumor analysis platform 161, a size prediction platform 175, a purity prediction platform 185, and/or a heatmap generation platform 195. The training tumor analysis platform 161, according to one technique, may create or receive one or more datasets of training data used to generate and train one or more machine learning models that, when implemented, conduct tumor analysis. According to one technique, the training tumor analysis platform 161 may include a plurality of software modules, including a training data intake module 162 and/or a training analysis module 163. The data and/or machine learning systems output by training tumor analysis platform 161 may be stored, e.g., in storage device 109, or used by other systems, e.g., size prediction platform 175, purity prediction platform 185, and/or heatmap generation platform 195.

Training data intake module 152, according to one aspect, may create or receive training data (e.g., biomarker prediction(s), embedding vector(s), the plurality of tiles, digital medical images, foreground data, background data, etc.) that may be used to train one or more machine learning systems for tumor analysis. The training data may be received from any one or any combination of server systems 110, physician servers 121, hospital servers 122, clinical trial servers 123, research lab servers 124, and/or laboratory information systems 125. Training data may come from real sources (e.g., humans, animals, etc.) or may come from synthetic sources (e.g., graphics simulators, graphics rendering engines, 3D models, etc.).

The training analysis module 163 may generate, using the training data as input, one or more machine learning systems capable of tumor analysis. In some examples, a third party may generate the one or more trained machine learning systems and provide the trained machine learning system(s) to server systems 110 for storage (e.g., in storage devices 109) and/or execution by tumor analysis system 104. Training analysis module 163 may train a graph neural network, a convolutional neural network, a transformer neural network, or any other suitable type of machine learning system for tumor analysis. Training analysis module 163 may store the biomarker prediction and the results of the tumor analysis, e.g., storage devices 109. Methods for training the one or more machine learning systems of training analysis module 133 are described herein.

According to one technique, the size prediction platform 175 may include software modules, such as a target data intake module 176, a sizing model 177, and an output interface 178. Size prediction platform 175, according to one aspect, may receive a request for a tumor size prediction for at least one digital medical image and/or the embedding vector(s), and execute one or more of the machine learning systems trained by training tumor analysis platform 161 for tumor analysis. For example, the request may be received from any one or any combination of the physician servers 121, hospital servers 122, clinical trial servers 123, research lab servers 124, and/or laboratory information systems 125.

Target data intake module 176, according to one aspect, may create or receive target data (e.g., biomarker prediction(s), embedding vector(s), plurality of tiles, images, optionally clinical data, etc.) that may be used as an input for one or more trained machine learning systems for tumor size prediction. For example, target data intake module 176 may receive embedding vector(s), which may be used as an input for one or more trained machine learning systems. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). The target data intake module 176 may create or receive the one or more datasets of target data, e.g., embedding vector(s). For example, the datasets may include one or more datasets corresponding to embedding vector(s) and/or, optionally, one or more datasets corresponding to biomarker prediction(s), digital medical images, and/or clinical data. In some examples, a subset of target data may overlap between or among the various datasets for images and/or clinical data. The target datasets may be stored on a digital storage device, e.g., one of storages devices 109.

Sizing model 177, according to one aspect, may be configured to analyze the embedding vector(s), e.g., to predict tumor size. Sizing model 177 may include any suitable machine learning systems, including but not limited to, graph neural networks, convolutional neural networks, transformer neural networks, etc. Sizing model 177 may execute the various machine learning systems generated by training tumor analysis platform 161, e.g., training analysis module 163, to facilitate the size prediction.

The output interface 178 may be used to output the size prediction(s) (e.g., to a screen, monitor, storage device, web browser, etc.). According to some techniques, output interface 178 may output the size prediction(s) for use as input in a subsequent process described herein. The size prediction(s) and other data produced or used by tumor analysis system 104 may be stored in one or storage devices 109.

Purity prediction platform 185, according to one aspect, may receive a request for a tumor purity prediction for at least one digital medical image and/or the embedding vector(s), and execute one or more of the machine learning systems trained by training tumor analysis platform 161 for tumor analysis. For example, the request may be received from any one or any combination of the physician servers 121, hospital servers 122, clinical trial servers 123, research lab servers 124, and/or laboratory information systems 125.

Target data intake module 186, according to one aspect, may create or receive target data (e.g., biomarker prediction(s), embedding vector(s), plurality of tiles, images, optionally clinical data, etc.) that may be used as an input for one or more trained machine learning systems for tumor purity prediction. For example, target data intake module 186 may receive embedding vector(s), which may be used as an input for one or more trained machine learning systems. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). The target data intake module 186 may create or receive the one or more datasets of target data, e.g., embedding vector(s). For example, the datasets may include one or more datasets corresponding to embedding vector(s) and/or, optionally, one or more datasets corresponding to biomarker prediction(s), digital medical images, and/or clinical data. In some examples, a subset of target data may overlap between or among the various datasets for images and/or clinical data. The target datasets may be stored on a digital storage device, e.g., one of storages devices 109.

Purity model 187, according to one aspect, may be configured to analyze the embedding vector(s), e.g., to predict tumor purity. Purity model 187 may include any suitable machine learning systems, including but not limited to, graph neural networks, convolutional neural networks, transformer neural networks, etc. Purity model 187 may execute the various machine learning systems generated by training tumor analysis platform 161, e.g., training analysis module 163, to facilitate the purity prediction.

The output interface 188 may be used to output the purity prediction(s) (e.g., to a screen, monitor, storage device, web browser, etc.). According to some techniques, output interface 188 may output the purity prediction(s) for use as input in a subsequent process described herein. The purity prediction(s) and other data produced or used by tumor analysis system 104 may be stored in one or storage devices 109.

Heatmap generation platform 195, according to one aspect, may receive a request for heatmap generation for at least one digital medical image and/or the embedding vector(s), and execute one or more of the machine learning systems trained by training tumor analysis platform 161 for tumor analysis. For example, the request may be received from any one or any combination of the physician servers 121, hospital servers 122, clinical trial servers 123, research lab servers 124, and/or laboratory information systems 125.

Target data intake module 196, according to one aspect, may create or receive target data (e.g., biomarker prediction(s), embedding vector(s), plurality of tiles, images, optionally clinical data, etc.) that may be used as an input for one or more trained machine learning systems for heatmap generation. For example, target data intake module 196 may receive embedding vector(s), which may be used as an input for one or more trained machine learning systems. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). The target data intake module 196 may create or receive the one or more datasets of target data, e.g., embedding vector(s). For example, the datasets may include one or more datasets corresponding to embedding vector(s) and/or, optionally, one or more datasets corresponding to biomarker prediction(s), digital medical images, and/or clinical data. In some examples, a subset of target data may overlap between or among the various datasets for images and/or clinical data. The target datasets may be stored on a digital storage device, e.g., one of storages devices 109.

Heatmap model 197, according to one aspect, may be configured to analyze the embedding vector(s), e.g., to generate at least one heatmap. The at least one heatmap may be a tile-level heatmap, a cell-level heatmap, etc. In one aspect, the tile-level heatmap may be generated based on the embedding vector(s), and the cell-level heatmap may be generated based on the tile-level heatmap. Heatmap model 197 may be configured to generate a display including one or both of the tile-level heatmap or the cell-level heatmap overlaid on the at least one digital medical image. Heatmap model 197 may include any suitable machine learning systems, including but not limited to, graph neural networks, convolutional neural networks, transformer neural networks, etc. Heatmap model 197 may execute the various machine learning systems generated by training tumor analysis platform 161, e.g., training analysis module 163, to facilitate the heatmap generation.

The output interface 198 may be used to output the generated heatmap(s) (e.g., to a screen, monitor, storage device, web browser, etc.). According to some techniques, output interface 198 may output the generated heatmap(s) for use as input in a subsequent process described herein. The generated heatmap(s) and other data produced or used by tumor analysis system 104 may be stored in one or storage devices 109.

FIG. 2 depicts a schematic 200 of an exemplary system for predicting biomarkers, according to one or more embodiments. As depicted in schematic 200, at least one digital medical image 202 may be received as an input via foundation model 137. The at least one digital medical image 202 may be received as a plurality of foreground tiles generated by image analysis system 101 of FIG. 1B. Returning to FIG. 2, foundation model 137 may be configured to analyze the at least one digital medical image 202, and/or the plurality of foreground tiles of the at least one digital medical image 202, to determine vector embeddings 204.

The vector embeddings 204 may be received as an input via at least one of sizing model 177, purity model 187, aggregator model 157, and/or heatmap model 197. Sizing model 177 may be configured to generate a tumor size prediction 208 based on vector embeddings 204. Purity model 187 may be configured to generate a tumor purity prediction 210 based on vector embeddings 204.

Aggregator model 157 may be configured to generate biomarker prediction 206 based on vector embeddings 204. Heatmap model 197 may be configured to generate at least one heatmap based on vector embeddings 204 and/or biomarker prediction 206. For example, heatmap model 197 may be configured to generate tile-level heatmap(s) 212 based on vector embeddings 204 and/or biomarker prediction 206. In a further example, heatmap model 197 may be configured to generate cell-level heatmap(s) 214 based on vector embeddings 204, biomarker prediction 206, and/or tile-level heatmap(s) 212.

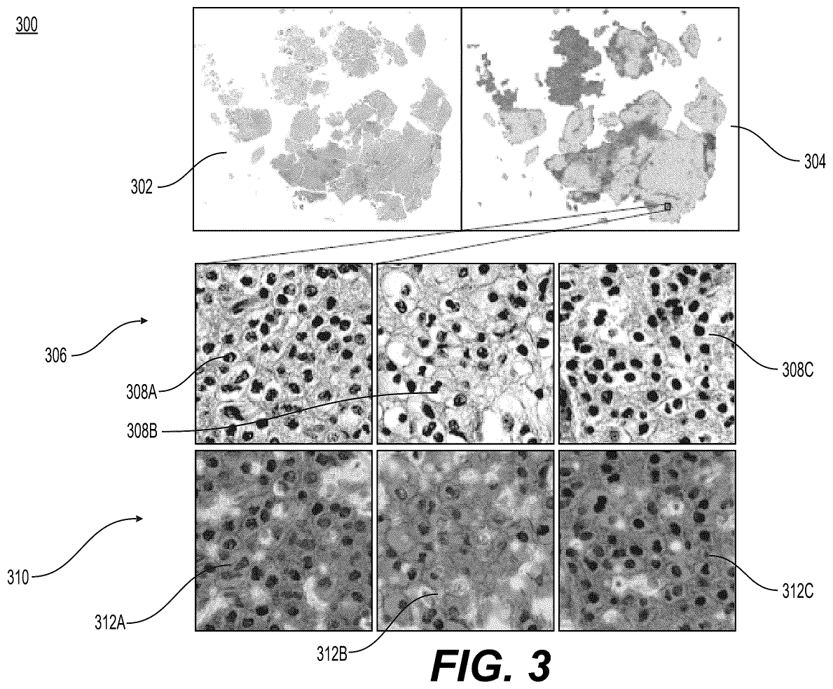

FIG. 3 depicts a schematic 300 of exemplary heatmaps 304, 310, according to one or more embodiments. Heatmaps 304, 310 may be attentional focus heatmaps of samples 302, 306. Samples 302, 306 may be digital medical images, tissue samples, etc. As depicted in FIG. 3, heatmap 312a may be generated based on sample 308a, heatmap 312b may be generated based on sample 308b, heatmap 312c may be generated based on sample 308c, etc.

The attentional focus heatmaps 304, 310 may indicate greater attention paid to relevant biomarkers, such as cytoplasmic clearing around the nucleus (e.g., characteristic of a formalin-fixed oligodendroglioma), at least one nucleus, etc. Heatmaps 304, 310 may be the result of the systems described herein advantageously refining biomarker signal(s) with key features that may appear subtle to a human pathologist.

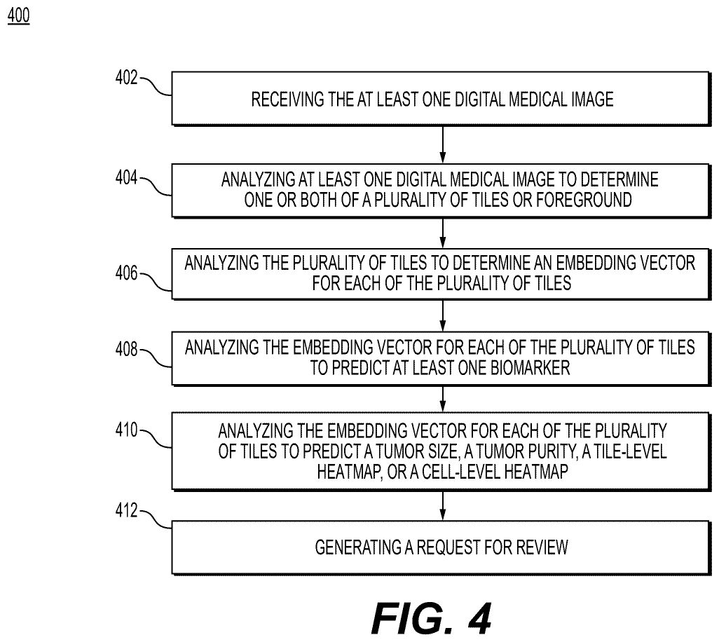

FIG. 4 depicts a flow diagram for an exemplary process 400 for predicting biomarkers, according to one or more techniques. At step 404, at least one digital medical image may be received (e.g., via image analysis system 101). At step 404, the at least one digital medical image may be analyzed to determine one or both of a plurality of tiles or foreground (e.g., via tile generation model 137, foreground detection model 138, etc.). For example, the at least one digital medical image may be analyzed to determine at least one image that includes foreground.

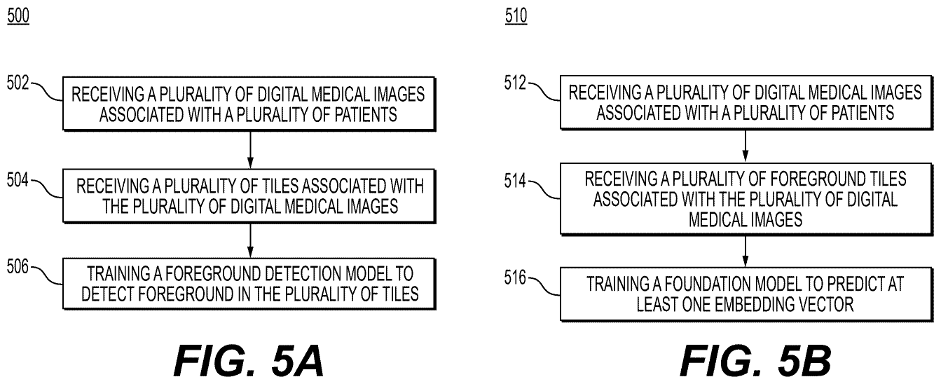

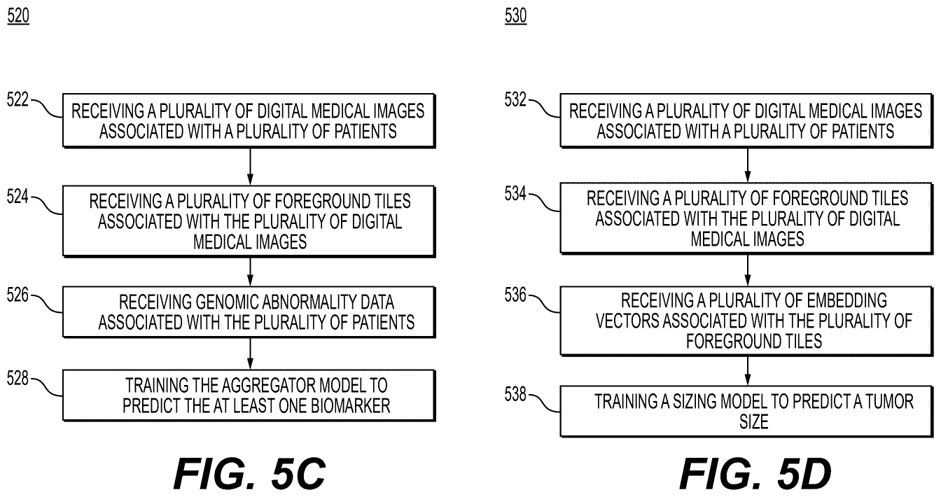

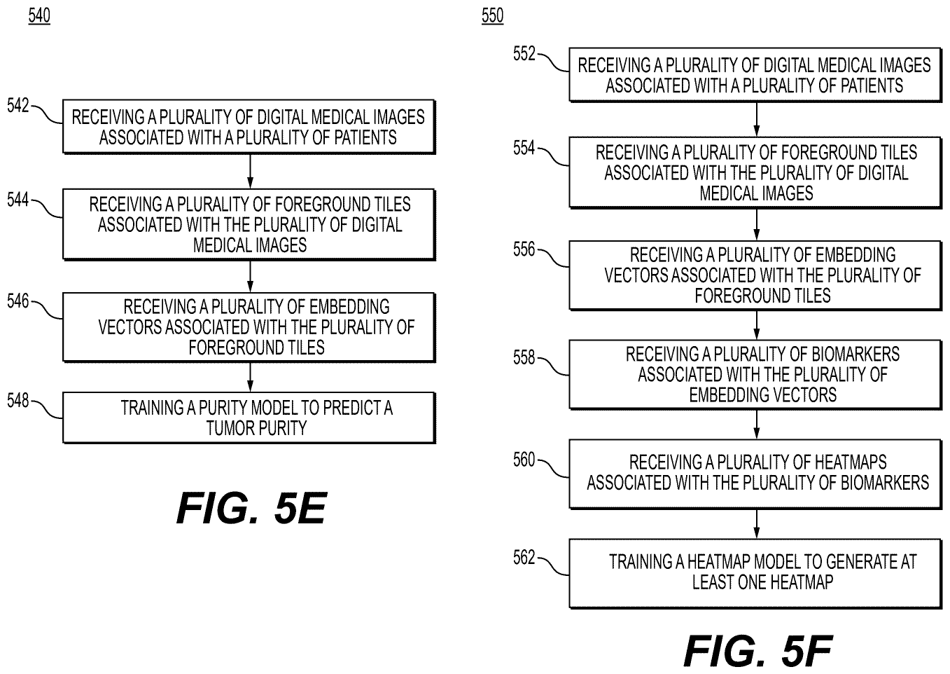

Foreground detection model 138 may be trained via method 500 of FIG. 5A. At step 502, a plurality of digital medical images associated with a plurality of patients may be received. At step 504, a plurality of tiles (e.g., a plurality of foreground tiles) associated with the plurality of digital medical images may be received. At step 506, a foreground detection model may be trained to detect foreground in the plurality of tiles based on the plurality of digital medical images and/or the plurality of tiles.

Returning to FIG. 4, at step 406, the plurality of tiles (e.g., the plurality of foreground tiles) may be analyzed to determine an embedding vector for each of the plurality of tiles (e.g., via foundation model 147). In one aspect, the embedding vectors may be predicted at a tile-level based on the plurality of digital medical images.

Foundation model 147 may be trained via method 510 of FIG. 5B. At step 512, a plurality of digital medical images associated with a plurality of patients may be received. At step 514, a plurality of tiles (e.g., a plurality of foreground tiles) associated with the plurality of digital medical images may be received. At step 516, a foundation model may be trained to predict at least one embedding vector based on the plurality of digital medical images and/or the plurality of tiles.

Returning to FIG. 4, at step 408, the embedding vector for each of the plurality of tiles may be analyzed to predict at least one biomarker (e.g., via aggregator model 157). The at least one biomarker may be predicted by aggregating the embedding vector for each of the plurality of tiles into at least one slide-level prediction. The at least one biomarker may include a first biomarker, a second biomarker, a third biomarker, etc. In one aspect, each of the first biomarker, the second biomarker, the third biomarker, etc. may be a different biomarker type. In one aspect, the at least one biomarker may be predicted based on genomic abnormality data associated with the plurality of patients.

Aggregator model 157 may be trained via method 520 of FIG. 5C. At step 522, a plurality of digital medical images associated with a plurality of patients may be received. At step 524, a plurality of tiles (e.g., a plurality of foreground tiles) associated with the plurality of digital medical images may be received. At step 526, genomic abnormality data associated with the plurality of patients may be received. At step 528, an aggregator model may be trained to predict the at least one biomarker based on the plurality of digital medical images, the plurality of tiles, and/or the genomic abnormality data.

Returning to FIG. 4, optionally at step 410, the embedding vector for each of the plurality of tiles may be analyzed to predict a tumor size, a tumor purity, a tile-level heatmap, and/or a cell-level heatmap. The tumor size may be predicted (e.g., via sizing model 177) based on the plurality of embedding vectors. Sizing model 177 may be trained via method 530 of FIG. 5D. At step 532, a plurality of digital medical images associated with a plurality of patients may be received. At step 534, a plurality of tiles (e.g., a plurality of foreground tiles) associated with the plurality of digital medical images may be received. At step 536, a plurality of embedding vectors associated with the plurality of foreground tiles may be received. At step 538, a sizing model may be trained to predict a tumor size based on the plurality of digital medical images, the plurality of tiles, and/or the plurality of embedding vectors.

The tumor purity may be predicted (e.g., via purity model 187) based on the plurality of embedding vectors. Purity model 187 may be trained via method 540 of FIG. 5E. At step 542, a plurality of digital medical images associated with a plurality of patients may be received. At step 544, a plurality of tiles (e.g., a plurality of foreground tiles) associated with the plurality of digital medical images may be received. At step 546, a plurality of embedding vectors associated with the plurality of foreground tiles may be received. At step 548, a purity model may be trained to predict a tumor purity based on the plurality of digital medical images, the plurality of tiles, and/or the plurality of embedding vectors.

The tile-level heatmap may be generated (e.g., via heatmap model 197) based on the plurality of embedding vectors and/or the biomarker prediction. The cell-level heatmap may be generated (e.g., via heatmap model 197) based on the plurality of embedding vectors, the biomarker prediction, and/or the tile-level heatmap. Heatmap model 197 may be trained via method 550 of FIG. 5F. At step 552, a plurality of digital medical images associated with a plurality of patients may be received. At step 554, a plurality of tiles (e.g., a plurality of foreground tiles) associated with the plurality of digital medical images may be received. At step 556, a plurality of embedding vectors associated with the plurality of foreground tiles may be received. At step 558, a plurality of biomarkers associated with the plurality of embedding vectors may be received. At step 560, a plurality of heatmaps (e.g., tile-level heatmaps, cell-level heatmaps, etc.) associated with the plurality of biomarkers may be received. At step 562, a heatmap model may be trained to generate a tile-level heatmap or a cell-level heatmap based on the plurality of digital medical images, the plurality of tiles, the plurality of embedding vectors, the plurality of biomarkers, and/or the plurality of heatmaps.

Returning to FIG. 4, optionally at step 412, a request for review may be generated. The request for review may be generated based on at least one of the plurality of digital medical images, the plurality of tiles, the plurality of embedding vectors, the plurality of biomarkers, and/or the plurality of heatmaps. The request for review may be transmitted to third-party device 124 (e.g., to be displayed via graphical user interface 125 of third-party device 124).

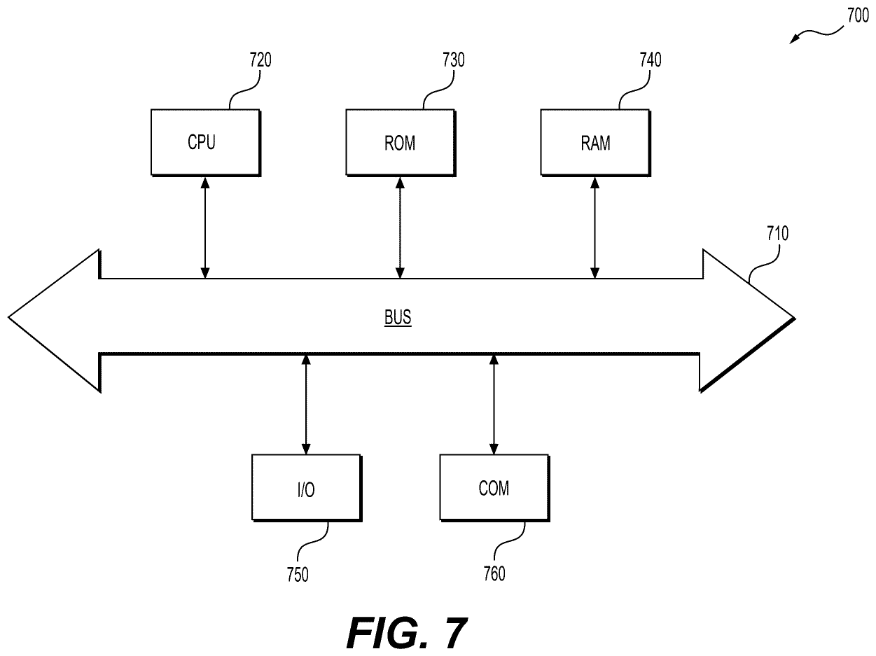

FIG. 7 illustrates an example system or device 700 that may execute techniques presented herein. Device 700 may include a central processing unit (CPU) 720. CPU 720 may be any type of processor device including, for example, any type of special purpose or a general-purpose microprocessor device. As will be appreciated by persons skilled in the relevant art, CPU 720 also may be a single processor in a multi-core/multiprocessor system, such system operating alone, or in a cluster of computing devices operating in a cluster or server farm. CPU 720 may be connected to a data communication infrastructure 710, for example a bus, message queue, network, or multi-core message-passing scheme.

Device 700 may also include a main memory 740, for example, random access memory (RAM), and also may include a secondary memory 730. Secondary memory 730, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 730 may include similar means for allowing computer programs or other instructions to be loaded into device 700. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 700.

Device 700 also may include a communications interface (COM) 760. Communications interface 760 allows software and data to be transferred between device 700 and external devices. Communications interface 760 may include a modem, a network interface (such as an Ethernet card), a communications port, a PCMCIA slot and card, or the like. Software and data transferred via communications interface 760 may be in the form of signals, which may be electronic, electromagnetic, optical or other signals capable of being received by communications interface 760. These signals may be provided to communications interface 760 via a communications path of device 700, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 700 may also include input and output ports 750 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

### Exemplary Results

### Digital Biomarker Panel Enables High-Throughput Pancancer Biomarker Screening

The system described herein may predict the likelihood of the presence of each of 1,228 genomic features derived from the IMPACT dataset (as described below for model development details). The model may be intended to screen for any genomic alteration among 505 genes to prioritize or guide further confirmatory sequencing.

By computing the average precision (AP) and area under the receiver operating characteristic curve (AUC) for 1,228 biomarkers in the test set, a positive correlation between AP and the ratio of positive samples of labels in the training set as expected (FIG. 3A). In contrast, there may have been no strong correlation between AUC and the ratio of positive samples. The sensitivity and specificity for biomarkers showing AUC>0.5 may be displayed below in FIG. 3B. The techniques described herein may have identified 655 genetic alteration biomarkers, representing 320 genes, in 44 cancer types that were above the threshold expected by chance, with an AUC>0.5, and at least three positive samples with a positive ratio>2%. The system may have observed variability in the accuracy of biomarker prediction across different cancer types, which can be attributed to differences in sample size and prevalence as displayed in FIG. 3C below.

By evaluating the performance of each biomarker in 7,208 samples (4,744 primary and 2,464 metastatic samples) of the fifteen most common cancer types treated, the system described herein may have been able to detect 391 genetic alteration biomarkers in the test set, based on the filtering criteria of AUC>0.75, sensitivity >0.75 and specificity >0.2. This set of biomarkers, with a mean AUC of 0.84 (mean sensitivity=0.92 and mean specificity=0.55), may represent 483 distinct gene and tumor histology associations, i.e., a signal in at least one cancer type for 222 genes. A prediction signal for TP53 oncogenic mutations may have been identified in fourteen of the most commonly treated cancer types, meeting or exceeding the filtering criteria used. The mean AUC may have been 0.86 (n=13, range 0.76-0.94) in primary samples and 0.84 (n=8, range 0.770.97) in metastatic samples. TP53 may have also detected in esophagogastric cancer, achieving a high AP of 0.77 in primary cancers (n=148 positive and n=77 negative samples), with a sensitivity of 0.98 and a specificity of 0.34. However, the AUC of 0.7 may have fallen below the filtering criterion of 0.75. Other commonly mutated oncogenes where high prediction AUCs for alteration were obtained in five or more of the most commonly treated cancer types may have included CDKN2A, CDKN2B, TERT, KRAS, CCND1, PTEN, RB1, PIK3CA, BRCA1, AGO2, ARID1A, CDK12, CTNNB1, ERBB2, KMT2B, MYC, NF1 as shown FIG. S3A. Overall, the AUCs for prediction of a genomic alteration may have been higher for WSIs obtained from primary tumors versus WSIs obtained from samples of metastatic lesions.

Among the fifteen most commonly treated cancer types, colorectal cancers may have had the highest number of genes with a model prediction AUC>0.75 on the test set (n=61 genes in primary and n=30 genes in metastatic samples), followed by endometrial cancers (n=61 in primary and n=19 in metastatic samples) as shown in FIG. S3B. Breast cancers (n=41 genes in primary and n=17 genes in metastatic samples), bladder cancers (n=26 genes in primary and n=21 genes in metastatic samples), and ovarian cancers (n=29 genes in primary and n=20 genes in metastatic samples) may have also showed notable numbers of genes with AUC>0.75. Since gliomas essentially never metastasize outside the central nervous system, no metastases may have been analyzed for this cancer type (n=20 genes in primary samples).

By assessing high-performing biomarkers (defined as biomarkers achieving AUC>0.85) with inclusion criteria of sensitivity >0.8 and specificity >0.3, 80 biomarkers (with at least 5% positive sample ratio), representing 47 genes, may have been identified in the fifteen most common cancers with a mean AUC of 0.89 (AUC range 0.85-0.99, mean sensitivity=0.93, mean specificity=0.66) (FIGS. 3D, S5A). Top-performing genes with the best AUC scores in primary cancers may have included FGFR3 (bladder), CDH1 (breast), MSH3 (colorectal), TP53 and APC (endometrial), KMT2D (esophagogastric), IDH1 (glioma), SMAD4 (hepatobiliary), AGO2 (melanoma), STK11 and EGFR (NSCLC), TP53 and KRAS (ovarian), CDKN2B (pancreatic), BAP1 (renal cell carcinoma), CDKN2A (p16INK4a) (soft tissue sarcoma), and CDKN2A (p14ARF) (thyroid).

To assess the model's generalizability, further validation may have been conducted on an independent external dataset where the database has n=9,340 as described below. The optimal operating threshold determined from the tune set of the development cohort may have been used to convert the likelihood of a genomic alteration in each sample to binary predictions, indicating the presence or absence of the genetic variant in a gene. Twenty-seven of the top performing genes identified from the test set may have been validated in the TCGA cohort, with a mean AUC of 0.87 (range 0.77-0.94, mean sensitivity=0.91, mean specificity=0.6), including FGFR3 (bladder), CDH1 and ERBB2 (breast), BRAF and RNF43 (colorectal), PTEN (endometrial), KMT2B (gastric), IDH and ATRX (glioma), STK11 and EGFR (non-small cell lung cancer), BRAF and NRAS (thyroid). TP53 may have been validated in both breast and endometrial cancers, and KMT2D was validated in both colorectal and gastric cancers.

Biomarkers Associated with Histologic Subtypes of Cancers

The model described herein may further be trained to detect genomic alterations and to diagnose specific histologic subtypes of cancers, particularly those in which certain genomic alterations are diagnostic or show high concordance with specific phenotypes. For each top performing genomic biomarker within a cancer type, the system was compared to the test set inference probabilities of the biomarker between the histologic subtypes. The analysis may have revealed forty genomic alteration predictions that were highly associated with specific histologic subtypes (Kolmogorov-Smirnov (KS) test adjusted p-value <0.01, and AUC>0.85).

The performance of these subtype-specific biomarkers in predicting the histologic subtype diagnosis within the withheld test set may further have been tested. Rather than using genomic alterations as the ground truth, performance may have been evaluated based on the actual cancer and subtype diagnosis assigned to each case. Specifically, it may have been examined whether the model's prediction of subtype-associated genomic alterations could accurately identify the diagnosis of the corresponding subtype. For example, CDH1 alteration has a strong phenotype-genotype correlation with the invasive lobular carcinoma subtype of breast carcinoma. The model prediction of CDH1 oncogenic mutation presence may have been able to diagnose breast invasive lobular carcinoma with an AUC of 0.93, sensitivity of 0.94, and specificity of 0.77. This association may have also been validated in the TCGA cohort, showing an AUC of 0.95. Similarly, within thyroid carcinomas, prediction of a RET oncogenic mutation by the model described herein may have achieved an AUC of 0.99 in diagnosing medullary thyroid cancer (MTC). Deleterious mutations of ARID1A, a member of the SWI/SNF chromatin remodeling genes, may have been common in clear cell and endometrioid ovarian cancers. The prediction of oncogenic mutation/deletion in ARID1A may have displayed an AUC of 0.85 and 0.93, respectively, for identifying ovarian clear cell carcinoma (OCCC) (n=22, sensitivity=0.82, and specificity=0.79) and endometrioid ovarian cancers (n=15, sensitivity=0.93, specificity=0.76). KMT2D deficiency may drive lung squamous cell carcinoma (LUSC) and the evaluation may have showed that the diagnosis of LUSC by the prediction of KMT2D oncogenic mutation/deletion achieved an AUC of 0.90, with sensitivity of 0.96 and specificity of 0.54.

In soft tissue sarcomas, prediction of MDM2 amplification may have identified well differentiated liposarcoma with AUC=0.93 (sensitivity=0.94, specificity=0.55), driven by the high prevalence of MDM2 amplification in this subtype. When predicting well-differentiated liposarcoma (n=17) with atypical lipomatous tumor (n=2) and dedifferentiated liposarcoma (n=35), it may have showed a performance of 0.85 sensitivity and 0.6 specificity (AUC=0.85). The diagnosis of dedifferentiated liposarcoma via MDM2 amplification prediction may have been validated in TCGA cohort, demonstrating an AUC of 0.84. Moreover, TERT oncogenic mutation prediction may have correctly predicted myxoid liposarcoma diagnosis with AUC=0.93, sensitivity=1 and specificity=0.29, while the prediction of RB1 loss showed an AUC of 0.86 for diagnosis of leiomyosarcoma with sensitivity of 0.9 and specificity of 0.67. Finally, among sarcomas, WT1 fusion prediction diagnosed desmoplastic small round cell tumor which canonically carries a EWSR1-WT1 fusion with AUC=0.998, sensitivity=0.76 and specificity=1.

In pancreatic cancers, MEN1 and DAXX may be frequently mutated in pancreatic neuroendocrine tumor. Prediction of oncogenic mutation/deletion in these two genes could diagnose pancreatic neuroendocrine tumor with AUC>0.97 (sensitivity=0.79 and 0.55; specificity=0.99 and 1, respectively), while KRAS oncogenic mutation prediction correctly identified pancreatic adenocarcinoma diagnosis with AUC=0.92, sensitivity=0.87 and specificity=0.85. In renal cell carcinoma, loss of function in VHL may be a hallmark of clear cell renal cell carcinoma (ccRCC), and the prediction of VHL oncogenic mutation may have correctly identified ccRCC with an AUC of 0.93 (sensitivity=0.95 and specificity=0.83). Renal angiomyolipoma, though low prevalence (n=5) in the test set, may have been accurately detected by TSC2 oncogenic mutation prediction, achieving an AUC of 0.94, sensitivity of 1 and specificity 0.73. Both GNAQ and GNA11 oncogenic mutations were highly associated with uveal melanoma (KS adjusted p-value <0.01), and prediction of genomic alteration in these genes in melanoma may have showed an AUC of 0.94 and 0.93, respectively, for diagnosis of uveal melanoma. In glioma, oligodendroglioma is genetically defined by an IDH mutation and 1p19q codeletion. The prediction of IDH1 oncogenic mutation diagnosed oligodendroglioma with an AUC of 0.89, sensitivity of 0.94 and specificity of 0.73. The performance may have remained comparable (AUC=0.89, sensitivity=0.89 and specificity=0.75) when combining oligodendroglioma (n=17) with anaplastic oligodendroglioma (n=11). Both ARTX and IDH1 may have been strongly associated with anaplastic astrocytoma (KS-test adjusted p-value <0.01), and the prediction of oncogenic mutations in these genes showed an AUC>0.86, for diagnosis of anaplastic astrocytoma (n=22).

Biomarkers Associated with Targeted Therapeutic Hotspots

The system's predictions of genomic alterations that are indicative of a response to corresponding FDA-approved drugs across different cancers may have been evaluated. A list of 54 treatment-associated genes with specific hotspot mutations reported in My Cancer Genome (MCG) and OncoKB focusing on the actionable targets that are FDA-recognized biomarkers (OncoKB therapeutic evidence level 1) or standard care biomarkers recommended by the National Comprehensive Cancer Network (NCCN) or other expert panels (OncoKB therapeutic evidence level 2). The therapeutic target ground truth may then be established accordingly for each sample in the test set, based on the presence or absence of these specific mutations in the treatment-associated genes. By assessing the performance of the biomarkers trained by the model in predicting these therapeutic targets, the analysis may have identified 58 clinically relevant biomarkers in the fifteen most common cancers with AUC>0.75, sensitivity >0.70 and specificity >0.20. For example, BRAF V600E mutations are commonly found in melanoma, glioma, thyroid, lung and colorectal cancers, and are actionable targets as a standard of care in a subset of patients with these cancers. The performance of BRAF oncogenic alterations in predicting BRAF V600E mutations showed an AUC of 0.93 in primary thyroid cancers, with sensitivity of 0.89 and specificity of 0.81, and an AUC of 0.96 in metastatic thyroid cancers, with sensitivity of 0.94 and specificity of 0.78. Though not all biomarkers had sufficient positive samples (mutation carriers) to be evaluated in metastatic cancers, the evaluation on primary samples showed that detection of BRAF oncogenic alterations achieved an AUC of 0.87 in primary melanoma, with sensitivity of 0.95 and specificity of 0.30, and an AUC of 0.91 in primary colorectal cancers, with sensitivity of 0.98 and specificity of 0.48.

In one aspect, a heatmap may show the prediction of specific therapeutic hotspots in target genes (Y-axis) across common cancer types at primary (P) and metastatic (M) lesions, with AUC>0.5. The AUCs may have been evaluated by using biomarker inference probabilities as prediction and presence or absence of specific hotspot mutations in a targeted gene as ground truth. Outlined highlighted boxes may display the targeted genes harboring the hotspot mutations detected by our AI model, which achieved an AUC>0.75, sensitivity>0.7, specificity>0.2, with at least 5 positive samples in the corresponding genetic alteration biomarkers (N=58) associated with 33 genes.

Trastuzumab may be approved for the treatment of early-stage HER2+ (ERBB2amplified) breast cancers. Prediction of ERBB2 amplification showed an AUC of 0.84 (sensitivity-0.91 and specificity=0.54) in primary breast cancers and an AUC of 0.77 (sensitivity=0.89 and specificity=0.29) in primary gastric cancers. In addition, Fam-Trastuzumab Deruxtecan-nxki may be approved for treatment of unresectable or metastatic non-small cell lung cancer (NSCLC), where activating mutations and amplification of ERBB2 are common mechanisms for upregulation of HER2 expression. The evaluation may have showed that the prediction of ERBB2 oncogenic amplification or mutation in metastatic NSCLC (n=9 positive samples and n=329 negative samples) had a sensitivity of 0.89 and specificity of 0.27 (AUC=0.77), which showed a similar performance in primary NSCLC (n=30 positive samples and n=751 negative samples) with higher specificity (AUC=0.78, sensitivity=0.87 and specificity=0.46). The NPV of NSCLC in both primary and metastatic samples were 99%, which potentially could identify patients that do not have ERBB2 amplification or mutation, hence would not benefit for HER2-targeted therapy. Similarly, an AUC of 0.77 with sensitivity of 0.93 and specificity of 0.34 was achieved in metastatic bladder cancers (n=14 positive samples and n=67 negative samples), though low prevalence, suggesting a subset of bladder cancer patients could be screened who might benefit from HER2-targeted therapy.

FDA-approved receptor tyrosine kinase inhibitors (TKIs) such as gefitinib, erlotinib, afatinib, and osimertinib may be indicated for patients with specific oncogenic mutations in EGFR in the setting of advanced or metastatic NSCLC. The model's performance for predicting the TKI-targetable EGFR mutations (p.L858R, p.T790M, exon 19 deletion and exon 20 insertion) may have achieved an AUC of 0.86 (sensitivity=0.94 and specificity=0.45) in primary NSCLC and an AUC of 0.75 (sensitivity=0.86 and specificity=0.5) in metastatic NSCLC. Sotorasib and adagrasib may be approved small molecule inhibitors targeting KRAS p.G12C carriers in NSCLC. The KRAS p.G12C is the most common KRAS variant found in NSCLC patients, and the model described herein may have achieved an AUC of 0.78 (sensitivity=0.96 and specificity=0.30) for detection of this variant in primary NSCLC samples.

Tepotinib may be another TKI approved in NSCLC and targets MET exon 14 skipping mutations, typically due to changes affecting RNA splicing. The performance of the MET oncogenic alteration model in predicting MET exon 14 deletion/splicing mutations may have showed an AUC of 0.80, sensitivity of 0.86 and specificity of 0.42 in primary NSCLC. Two other genes with targeted therapies available in treatment may have been identified well by the model (AUC>0.75). ALK fusion detection in primary NSCLC had AUC=0.76, sensitivity=0.78, specificity=0.52 while ROS1 fusion detection in primary NSCLC had AUC=0.75, sensitivity=0.75, specificity=0.52. Both ALK1 and ROS1 fusion detection may result in primary NSCLC showed a high NPV of 0.99. Elacestrant may be approved by the FDA for patients with ER+, HER2−, and ESR1 mutated metastatic breast cancer. Prediction of ESR1 hotspot mutations (including D538, E380, L536, S463P, Y537), using the model described herein trained on ESR1 oncogenic mutations, may have achieved an AUC of 0.85 (sensitivity=0.86, specificity=0.62) in primary breast cancer, and an AUC of 0.76 (sensitivity=0.90, specificity=0.34) in metastatic breast cancer.

Other therapeutic targets for ER+/PR+ and HER2− patients with locally advanced or metastatic breast cancer may include PIK3CA, AKT1, and PTEN alterations. Prediction of PIK3CA hotspot mutations may have achieved a mean AUC of 0.8 (range 0.76-0.86) in five cancers with different prevalence of PIK3CA. Primary breast cancer may have had a high prevalence of PIK3CA mutation (n=182, 31%), and the model may have demonstrated an AUC of 0.76 (sensitivity=0.95, and specificity=0.32) predicting a PIK3CA mutation in this setting. Prediction of PIK3CA mutants in metastatic colorectal cancers (n=26, 11%) and primary NSCLC (n=35, 4%) may have had similar performance as breast cancer, showing an AUC of 0.76. The model may have showed better prediction of PIK3CA in ovarian cancers, with AUC=0.86 (n=13, 8%) for PIK3CA mutation detection in primary ovarian tumors and AUC=0.82 (n=12, 5.8%) for PIK3CA mutation detection in metastatic lesions. In primary soft tissue sarcoma, PIK3CA alterations were primarily PIK3CA amplification (n=8, 3%); however, the model may have showed an AUC of 0.82 for prediction of PIK3CA alterations in this setting. Compared to PIK3CA, PTEN loss may have had a lower prevalence in the training and test sets in lung and ovarian cancers. Despite this, high performance by the model for PTEN loss prediction may have been displayed in primary NSCLC (AUC=0.83, n=12, 1.5%) and ovarian cancers (AUC=0.9, n=8, 5%). In histologic settings with high prevalence of PTEN loss, such as endometrial cancer (n=192, 56%), high performance for PTEN loss prediction by the model may have been seen as well in primary and metastatic examples (AUC of 0.88 and 0.86, respectively). An AUC of 0.84 and 0.76 may have been achieved for detection of PTEN-mutant cases in primary colorectal and prostate cancers, respectively. Lastly, AKT1 (E17K) hotspot mutation may have been identified in primary colorectal cancers by the model with an AUC of 0.76, sensitivity of 0.89 and specificity of 0.37.

Erdafitinib may have been the first targeted therapy approved by FDA for the treatment of locally advanced or metastatic urothelial carcinoma with FGFR3 alterations. The model's prediction of FGFR3 hotspot mutations (R248C, S249C, G370C, Y373C) may have generated an AUC=0.88 (sensitivity=0.95 and specificity=0.48) in primary bladder cancer, slightly better than its performance in metastatic bladder cancers (AUC=0.79, sensitivity=0.73 and specificity=0.77). Prediction of oncogenic mutation in mismatch repair (MMR) genes such as MLH1, MSH2, and MSH6 may have achieved a mean AUC of 0.85 (range 0.84-0.87) in primary endometrial cancer, a mean AUC of 0.94 (range 0.91-0.95) in primary colorectal cancer, and an AUC of 0.94 in primary bladder cancer, respectively. Other genes associated with “hypermutator” phenotypes, such as POLE mutations, could also be detected by the model described herein. The model's prediction of POLE mutations in endometrial cancer may have achieved an AUC of 0.87, with a sensitivity of 0.86 and a specificity of 0.69.

Estimates of cost savings to enroll a 500-patient study, with all patients harboring a mutation in a particular gene by definitive NGS or polymerase chain reaction (PCR), after removing those cases unlikely to harbor a mutation in the gene based on pre-screening with the model described herein may have showed substantial cost savings across all genes and cancer types investigated as displayed in FIG. 7 below. Cost savings may have been highest for genes in which the targeted mutation type was lower in prevalence, due to reduced numbers of mutation-negative patients sent on for definitive molecular screening. On average, a 14% and 22% cost saving could be achieved in PCR (range 3%-27%) and NGS (range 11%-34%) testing, respectively. This translates to substantial cost reductions for PCR and NGS testing.

Biomarkers Associated with Signaling Pathways and Genome Instability

In addition to the genetic alterations in single genes, the capabilities of the model described herein may detect genetic alterations in any of a group of related genes canonically participating in a shared signaling pathway, hypothesizing that mutation in any member of the pathway may create an overlapping, shared phenotype due to the shared signaling cascade. In support, the model may have predicted genomic alterations in any of the canonical signaling pathways, e.g., receptor tyrosine kinase (RTK) MEK/ERK, mTOR, and TGF-β signaling pathways, as well as the homologous recombination deficiency (HRD) and DNA damage response (DDR) pathways. Genome instability may include tumor mutation burden high (TMB-H), microsatellite instability high (MSI-H) or deficient mismatch repair (dMMR), and chromosomal instability (CIN) measures: fraction of genome altered (FGA) (e.g., ≥30%), loss-of-heterozygosity (LOH) (e.g., ≥50%), genome instability (GI) index (e.g., ≥0.2), tetraploidy and whole genome doubling (WGD).

The MEK/ERK signaling pathway investigated may have included 34 genes including ALK, EGFR, ERBB2, FGFR1/2/3/4, RET and ROS1 and alterations in any of the pathway members may have been identified with an AUC of 0.83 (sensitivity=0.88 and specificity=0.25) in primary thyroid cancer, and an AUC of 0.88 (sensitivity=1 and specificity=0.23) in metastatic thyroid cancer. Other canonical signaling pathways could be similarly detected. For example, in primary endometrial cancers, the model may have achieved a mean AUC of 0.80 (range 0.77-0.83) in predicting genomic alterations in canonical mTOR signaling (n=16 genes including AKT1/2/3, MTOR, PIK3CA, PTEN and TSC1/2), homologous recombination deficiency (HRD) (n=11 genes, including BRCA1/2, ATM and PALB2), the TGF-β canonical signaling pathway (SMAD2/3/4 and TGFBR1/2), and the DNA damage response (DDR) (n=23 genes, including ATM, ATRX, BRCA1/2, MDM2/4 and PPP2R1A), with sensitivity and specificity in a range of 0.81-0.97 and 0.22-0.59, respectively. Phenotypes predictive of canonical signaling pathway alterations may have been found in other histologies, and in both primary and metastatic settings as well. For example, predicting genetic alterations in any of the canonical TGF-β signaling pathway genes may have shown an AUC of 0.88 in primary hepatobiliary cancer, with sensitivity of 0.89 and specificity of 0.53, while prediction of genomic alterations in any of the canonical HRD associated genes achieved 0.81 AUC for metastatic soft tissue sarcoma.

Genomic instability may be a hallmark of many cancers. Three measures of genomic instability may be examined herein: tumor mutation burden (TMB), microsatellite instability (MSI) and chromosomal instability (CIN), for model training and evaluation to see if phenotypes indicative of these measures of genomic instability could be detected.

Prediction of tumor mutation burden-high (TMB-H) may have achieved a mean AUC of 0.85 (range 0.76-0.9) in seven cancers, of which the prediction in primary endometrial and colorectal cancers showed the best performance with an AUC of 0.9 (sensitivity >0.9 and specificity >0.5), followed by esophagogastric cancer with an AUC of 0.86 in both primary and metastatic lesions. TMB-H prediction may have resulted in an AUC of 0.79 (sensitivity=0.89 and specificity=0.49) in primary NSCLC, where 150 samples harbored TMB-H and 624 had low TMB. TMB-H may be a rare molecular subgroup in soft tissue sarcoma and prostate cancer. Though the majority of soft tissue sarcoma had low TMB, a small proportion of primary samples harboring TMB-H (n=8, out of 276, 2.9%), may have been identified, with sensitivity of 1 and specificity of 0.31 (AUC=0.87), suggesting potential clinical and therapeutic implications. Similarly, prediction of TMB-H may have shown an AUC of 0.81 in primary prostate cancer (n=8, out of 326, 2.5%), with sensitivity of 0.88 and specificity of 0.5.

Microsatellite instability high (MSI-H) may have been found in 13.5% of primary colorectal cancer (n=84 MSI-H and n=538 MSS) and 18.6% (n=57 MSI-H and n=250 MSS) of primary endometrial cancer, from which prediction of MSI-H obtained an AUC of 0.98 (sensitivity=0.9 and specificity=0.95) and an AUC of 0.89 (sensitivity=0.89 and specificity=0.78) in primary colorectal and endometrial cancers, respectively. MSI-H may be less frequent in bladder cancer (n=8 out of 291, 2.7%), and the model may have shown an AUC of 0.98, sensitivity of 1 and specificity of 0.91.

The performance of detecting dMMR may have been evaluated, defined as loss of IHC staining in MMR (MLH1, MSH2, MSH6 and PMS2) proteins and/or presence of genetic alterations in MMR genes. dMMR may have been found in 13.5% of primary colorectal cancers (n=36 out of 267), 18% of primary endometrial cancer (n=27 out of 149), and 17% of primary gastric cancers (n=12 out of 71). The model may have achieved an AUC of 0.997 (sensitivity=1 and specificity=0.93) for detection of dMMR among primary colorectal cancers, an AUC of 0.94 (sensitivity=0.96 and specificity=0.71) in primary endometrial cancers, and 0.999 (sensitivity=1 and specificity=0.98) in primary gastric cancers. In addition, individuals with Lynch syndrome (LS) may be at increased hereditary risk of developing cancers with MSI-H/dMMR. The diagnosis of LS may be based on the detection of a germline pathogenic mutation in one of MMR genes or in EPCAM. A surrogate ground truth for Lynch Syndrome may be defined as the presence of germline mutation in any of MMR genes or EPCAM. Trained with this surrogate ground truth, the model may have shown a mean AUC of 0.87 (range 0.85-0.89) for prediction of LS in primary bladder, colorectal and endometrial cancers.

Chromosomal instability (CIN) may have been predicted by five metrics defined as presence of tetraploidy, whole genome doubling (WGD), fraction of genome altered (FGA)≥30%, loss-of-heterozygosity (LOH)≥50%, and genome instability index (GI index)≥0.2. CIN data may have been used for breast and high-grade serous ovarian cancer (HGSOC). In an exemplary use, when using tetraploidy as the ground truth for CIN, 178 samples were positive for tetraploidy, while 296 samples were negative for diploidy. From this use case, the model may have resulted in an AUC of 0.88 for CIN in primary breast cancers defined by identification of tetraploidy. WGD may be highly associated with tetraploidy. Using WGD as the definition of CIN, 100 samples may have been positive for WGD while 284 samples were negative for lacking WGD, from which the model obtained an AUC of 0.88 CIN defined by WGD among primary breast cancers. Similarly, the model may have been trained with FGA≥30% as ground truth resulted in an AUC of 0.91 from 313 samples positive for FGA≥30% and 192 samples with less than 30% of genome altered among primary breast cancers in evaluation, and LOH≥50% as ground truth resulted in an AUC of 0.87 in primary breast cancers where 27 samples had LOH≥50% and 478 samples were negative, i.e. less than 50% of genome harboring LOH. GI index may have been derived by incorporating both FGA and LOH. When using GI index ≥0.2 as ground truth, the model may have obtained an AUC of 0.90 in primary breast cancers, of which 233 had GI index of >0.2, and 272 samples had a GI index less than 0.2. Overall, in an exemplary use case, the CIN measures in primary breast cancer achieved a mean AUC of 0.89 (range 0.87-0.91), with sensitivity >0.9 and specificity >0.52, and the results in primary breast cancers may have been superior to model prediction in metastatic breast cancers overall for all definitions of CIN examined, with a mean AUC of 0.85 (range 0.83-0.9), sensitivity >0.89 and specificity >0.42.

In HGSOC, the CIN measures may have showed a mean AUC of 0.78 (range 0.66-0.85) in primary cancers and a mean AUC of 0.77 (range 0.63-0.91) in metastatic cancers. In primary HGSOC, only three CIN measures, FGA≥30% (AUC=0.85, n=78 positive, and n=11 negative samples), GI index ≥0.2 (AUC=0.84, n=73 positive and n=16 negative samples) and tetraploidy (AUC=0.78, n=54 positive and n=29 negative samples) passed the baseline criteria of the model performance for AUC>0.75, showing sensitivity >0.9 and specificity >0.44. In metastatic cancers, FGA≥30% and GI index>0.2 may have passed the baseline, showing an AUC of 0.91, sensitivity >0.89 and specificity >0.63.

### DISCUSSION

A wide range of genomic abnormalities may be documented in localized and advanced solid tumors via pan-cancer analysis. Genomic alterations in human cancers may arise from mechanisms ranging from loss of heterozygosity, activating and inactivating point mutation, chromosomal loss or gain, gene amplification, insertions and/or deletions of small or large portions of genes, splice site alterations, to epigenetic mechanisms such as hypermethylation of promoter regions or the gene itself. Detection of genomic alterations may plan an important role in diagnosis, therapy selection and response prediction. The mechanisms underlying carcinogenesis may not be necessarily silenced by therapy either; point mutations and epigenetic alterations may be common drivers of acquired tumor drug resistance. Genomic biomarkers may be utilized for prognosis and treatment prediction. The drive towards personalized medicine and delivery of targeted therapies may require robust biomarker assays to guide therapy selection in routine clinical care, and novel biomarker assays may often be developed to guide inclusion in randomized clinical trials for investigational targeted therapies. Central to this paradigm may be the ability to detect relevant genomic features efficiently and reliably. Specific genomic abnormalities may confer distinctive phenotypes, with particular biological characteristics and important clinical implications. The morphologic features of these phenotypes may be detectable by light microscopy by expert pathologists, such as the distinctively discohesive single file or single cell appearance, with intracytoplasmic lumens, of invasive lobular breast cancer that results from e-cadherin [CDH1] bi-allelic inactivation. However, morphological features of some genomic alterations may be too subtle, even for expert pathologists, to be discovered and reported—or routinely identified. The system described herein may implement machine learning techniques on digitally scanned and rendered WSIs to leverage the recent and significant advantages of computer-assisted analysis of digital images to identify sometimes subtle, sometimes new, features. Training to recognize relevant morphologic features for this kind of phenotype-genotype correlation modelling may require data from detailed molecular analyses to define specific alterations, in sufficient volume of cases, and in cases where scanned WSIs are available. Performing such training to generate, via machine learning, a new AI model that can rapidly and at scale in slide volume, scale in mutation targets, and scale in tumor histologies trained is possible and could lead to digital biomarkers to screen for clinically relevant genomic alterations in a way that saves time, tissue and costs for researchers and clinicians.

The system described herein may implement an artificial intelligence (AI)-based model utilizing ground truth histological and molecular data. The system may be a multi-label classifier for the prediction of 1,228 most clinically relevant genomic abnormalities in 505 genes from H & E WSIs in 70 human cancers. Evaluations of the model may have focused on the fifteen most common broad cancer types treated, and identified the most frequent mutations across histologies. The performance of the model may have been validated based on validation data. The performance of the model in TCGA validation dataset was slightly inferior to that observed in the internal validation. Slight divergence in validation results on TCGA data and images versus other validation sets is not uncommon in pathology image analysis development, for a variety of reasons. The main advantage of the TCGA dataset for these applications is that the data is external, public, and multi-institution, making it convenient to evaluate digital pathology image applications; however, TCGA was not developed with image analysis in mind. The primary purpose was to create an atlas of genomic alterations across cancers, and the digital image submission requirement was a fortunate afterthought. This meant permitting a “representative image” in TCGA which can vary dramatically in quality, coupled with variation in the sequencing method and analysis for results appended to the case. The internal dataset was on withheld clinical grade material and WSIs, with prior quality control on both the clinical staining done on the slide as well as on the resulting scanned image, and single institution sequencing results using an FDA-approved NGS method. Thus, the differences between the validation sets in terms of purpose, quality control, and ground truth development most likely explain the minor divergence in validation testing results.

Internal and external validation, despite some of the limitations, did confirm high performance in many aspects for the model described herein, with clinical and future research implications. For example, the prediction of MSI-H/dMMR associated genes showed strong performance in colorectal and endometrial cancers, as well as bladder and gastric cancers. Tumors with MSI-H/dMMR may often harbor high TMB and performance detecting TMB-H may have been best in colorectal and endometrial cancers. Even in tumor histologies with low prevalence of TMB-H, such as soft tissue sarcoma and prostate cancer], model described herein was able to identify the rare tumors in low prevalence histologies with TMB-H, suggesting the potential to identify patients who might respond to immune checkpoint inhibitor (ICI) therapy based on TMB in these less frequently screened histologies. Moreover, the model demonstrated strong performance in genomic biomarkers already routinely screened clinically for therapy selection and response prediction, such as targeted therapy associated genes (EGFR, KRAS, MET, ALK, and ROS1) in NSCLC, ERBB2 amplification in breast and gastric cancers for HER2-targeted therapy, FGFR3 genomic alterations to target FGFR-altered urothelial carcinoma, and BRAF oncogenic mutation targeting BRAF V600E mutation carriers in metastatic thyroid cancer, melanoma and colorectal cancers. This may suggest an AI assisted digital biomarker on the H & E stained WSI could be developed as a cost, time, and tissue sparing triage for definitive molecular testing for these targets. While some of the other genes investigated do not show a high AUC when set for a high sensitivity >0.9 screening assay to identify targeted mutation carriers, the results for genomic alteration prediction in genes like ALK/ROS1 in NSCLC and ERBB2 alterations in NSCLC may have shown a high NPV of 0.99. This may suggest that even these gene targets could be further developed as screening digital biomarkers to triage downstream definitive testing, functioning essentially as a highly accurate “rule out” assay, based on the NPV and thus ability to identify cases that almost certainly do not harbor one of these targeted mutations, and thus may not need definitive confirmatory testing.

An exemplary projected cost savings may be described using the performance characteristics results with the model described herein to identify BRAF mutations in melanoma and colorectal carcinoma, KRAS mutations in NSCLC and colorectal carcinoma, EGFR and MET mutations in NSCLC, and FGFR3 mutations in bladder cancer. It may have been demonstrated the use of the model as a digital biomarker to triage downstream NGS and PCR testing in these settings could potentially save an average of $200K (3%-27% cost reduction) for PCR testing and $800K (11%-34% cost reduction) for NGS testing, assuming a planned enrollment of at least 500 patients with the targeted mutation in these settings, with published prevalence estimates displayed in FIG. 7 below. Cost savings may have been most pronounced for low prevalence targets, and show that triage with AI models for digital biomarkers may be sufficient to remove cost barriers to clinical studies seeking to use targeted therapies in settings where the targeted mutation is low prevalence. Use cases for digital screening and cost savings can be extrapolated for other targets investigated, and beyond clinical trial and cost savings to cost reduction for routine clinical testing in particular laboratory and practice settings, when used to triage for more expensive definitive tests.

The model may have detected many genomic alteration targets in multiple histologies. For example, phenotypes predictive of genomic alterations in PIK3CA and PTEN were identified in several tumor histologies. There may be several active clinical trial programs for drugs targeting these genes, including pan-solid tumor trials or cohorts. The ability of the model described herein to identify genomic alteration signals in multiple histologies may have use in research applications, ranging from cost-effective screening of tissues and tissue blocks to find uncommon or rare genomic alterations within histologies (i.e., TMB-high cases of prostate cancer), identification of phenotypes predicting the same forms of genomic alterations across several histologies for hypothesis generation, including models predicting shared phenotypes resulting from activating or inactivating mutations in members of canonical signaling pathways, as we demonstrated in our results. Screening for genomic alterations across histologies may also be utilized in clinical trial settings to rapidly and cost-effectively identify patients who could be candidates for a clinical study, before investing significant time, tissue, and resources into definitive molecular testing. This may not only speed up drug development, but better match patients to clinical trials which are appropriate for their particular tumor.

It may be expected that genomic alterations with known strong phenotype correlations in subtypes of certain histologies to be biased towards these subtypes in outputs from the model. As expected, MDM2 amplification detection in soft tissue sarcoma with AUC of 0.84 when all sarcomas were tested on subtype analysis may have showed primarily recognition of the distinctive liposarcoma phenotype with AUC of 0.93. Similarly, strong results for VHL when all renal cell carcinomas were evaluated may have been biased to detection of clear cell renal cell carcinoma, a distinct histologic subtype that is known to be driven by VHL loss. The top performing gene prediction in all gliomas may have been IDH1 (AUC of 0.93), which may have been driven by detection of oligodendroglioma, which is genetically defined by IDH mutation. This may demonstrate that the model and approach, when a strong correlation of phenotype with specific genomic alterations is expected in particular diagnoses and subtypes, finds the expected phenotype-genotype correlation, and the unsupervised model may be training itself to known phenotypes in settings with known phenotype-genotype correlations. Identification of these expected biases in subtype diagnosis detection in settings where phenotype and genotype were already known to be correlated, across histologies we tested, may strongly suggest that the model is not being spurious in identifying high confidence prediction of genomic alterations, even in genes where previous phenotypes have not been identified and described by human pathologists. Thus, coupled with high confidence detection of genomic alterations in settings where high prevalence actionable targets are also previously described, the finding of expected diagnostic phenotype-genotype correlations shows the method and model here may be robust across genes and histologies.

The model described herein may thus be used to support pathologists and researchers to accurately predict gene mutations based on subtle morphological features reflecting these genomic alterations, and facilitate the diagnosis of histologies harboring disease-defining genetic alterations, or drive hypothesis about the novel phenotypic-genotypic correlation our model is discovering. Clinical application of such digital assays may requires high performance, however, this could be further improved through re-training with larger patient numbers in focused histologic and clinical settings. This could potentially eliminate the need to examine additional, precious tumor tissue for further analyses such as IHC, Fluorescence In Situ Hybridization (FISH) or NGS testing to assess genomic alterations, thereby saving time, costs and tissue. Moreover, identifying targets important for clinical trials could facilitate the rapid development of digital biomarkers for tissue-preserving screening, enabling quicker identification of patients eligible for enrollment and further optimizing resources in clinical trial screening.

Additionally, digital biomarkers could be used to pre-select cases most likely to harbor specific genomic alterations, justifying the costs associated with definitive genetic analysis. Such digital biomarker molecular predictions may enable future fusion of clinical variables such as histologic grade/stage, ER/PR status in breast cancers, Gleason score in prostate cancers, and prior/post treatment status with predicted genomic abnormalities for multi-modal refinement of treatment algorithms with future research.

The limitations may include the following factors: data and analyses have been restricted to WSIs taken from a cohort which included a small number of germline variants. Therefore, it may yet to be determined if the observations will generalize to other sequencing assays. For instance, other assays may not employ a germline control to whether variants are germline or somatic, which can affect the interpretation of the sequencing results.

The performance of model may vary when applied to publicly available datasets, due to the fact that the sequencing for these cohorts was performed in a research setting with research approaches for library preparations and was not performed to a clinically meaningful sequencing depth.

Finally, some of the genomic alterations studied may have been present in relatively small numbers of cases in the training dataset, due to limited prevalence in the cases available. Adding additional cases with these alterations may reveal a stronger signal. The development cohort data may be enriched in primary samples compared to metastatic samples, which may explain why better performance was typically observed in primary samples vs metastatic samples across the histologies tested. Collecting more samples from metastatic lesions may uncover more biomarkers linked to metastasis, and/or stronger performance of genomic alteration predictions in metastatic lesions.

The method described herein may be automated for rapid high-throughput AI-assisted screening of cancers not only to detect clinically meaningful genomic abnormalities across histologies, but also to identify the histologies characterized by genomic alterations, capable of directing selection of patients for definitive genomic analysis and/or clinical decisions. This pipeline may be cost and time efficient, tissue sparing and capable of application to large patient populations with reliable performance metrics. This approach may be suitable for application in research, including clinical trial settings, across different types of cancer.

### Exemplary Methods

### Patient Cohorts, Histopathologic and Genomic Analyses

A sample may be defined as an assay paired with one or more H & E stained WSIs taken from the same formalin-fixed paraffin-embedded (FFPE) tissue block. The exemplary dataset used herein (e.g., IMPACT) may include 43,605 samples (47,960 WSIs) from 71 cancer types (70 cancer types and a “Unknown” category for which the cancer type is not known). 28,351 (65%) of samples may be primary cancer sites while the remaining 15,254 (35%) may be metastasis samples. All image data may have been retrieved from a hospital archive and verified to meet staining quality standards for histopathology review. All biopsy glass slides may have been scanned with Leica Aperio AT2 scanners (Leica Biosystems, Division of Leica Microsystems Inc, Buffalo Grove, IL, USA) at 20×(0.5 microns per pixel) or 40×(0.25 microns per pixel) magnification. The cohort may have underwent paired tumor-normal targeted sequencing using the IMPACT assay. Bioinformatic pipelines may have been employed to analyze the sequencing results. The analysis may have determined the genomic alteration status, including mutations (single-nucleotide variants (SNVs) and insertion/deletions (indels)), copy number variations (amplification and deletions), and structural rearrangements (fusions) in 505 important cancer associated genes. The list of cancer associated genes may have been compiled from four IMPACT versions including v3, v5, v6 and v7 that respectively encompass 341, 410, 468 and 505 genes. Additional fusion events confirmed by a Fusion panel via RNA sequencing may have been incorporated to improve the coverage of fusion detection for the genes that are available from IMPACT panel. The annotation of the oncogenicity and clinical implication of specific genetic alterations may have been determined using OncoKB. Additionally, dMMR status may have been confirmed by IHC assay.

Among the exemplary IMPACT panel genes, five groups of genes that are canonically participated in shared pathways associated with DNA repair mechanisms may have been identified. The gene members of each pathways may have been retrieved. DNA damage response (DDR) may include 23 genes: ATM, ATR, ATRX, BRCA1, BRCA2, BRIP1, FANCA, FANCC, FANCD2, FANCE, FANCF, FANCG, FANCL, MDM2, MDM4, MLH1, MUTYH, NPM1, PALB2, PPP2R1A, RAD50, RAD51, STAG2; receptor tyrosine kinase (RTK) pathway of 34 genes ALK, CBL, CSF1R, DDR2, EGFR, EPHA3, EPHA5, EPHB1, ERBB2, ERBB3, ERBB4, FGF19, FGF3, FGF4, FGFR1, FGFR2, FGFR3, FGFR4, FLT1, FLT3, FLT4, HGF, IGF1R, KIT, MET, NF1, NTRK1, NTRK2, NTRK3, PDGFRA, PDGFRB, PTPN11, RET, ROS1; homologous recombination deficiency (HRD) pathway of 11 genes: BRCA1, BRCA2, PALB2, ATM, CHEK1, CHEK2, RAD51, FANCA, CDK12, RAD51B, RAD51C; mTOR signaling pathway of 16 genes: AKT1, AKT2, AKT3, CRKL, IRS2, MTOR, PIK3CA, PIK3CG, PIK3R1, PIK3R2, PTEN, RICTOR, RNF43, RPTOR, TSC1, TSC2; and TGF-β signaling pathway of 5 genes: SMAD2, SMAD3, SMAD4, TGFBR1, TGFBR2.

The external validation dataset may include samples from TCGA PanCancer Atlas project. A total of 11,406 diagnostic WSIs, corresponding to thirty two cancer types, may have been retrieved together with the genomic alteration data, including mutation, copy number aberration, and fusion, from TCGA and the cBioPortal, respectively. 36 slides may have been excluded from the validation cohort due to missing microns-per-pixel (mpp) information and a further three slides may have been excluded due to being out of focus and no foreground tiles being detected. The remaining 9,340 samples (11,367 WSIs) may define the validation cohort.

### Development of an AI-Based System for the Detection of Digital Biomarkers in Pan-Cancer Cancer Using Whole Slide Images

The pan-cancer digital biomarker screening model described herein may be configured to predict genomic abnormalities of interest in human cancers from H & E WSIs. The model may have been trained on 33,564 diagnostic clinical WSIs from a cohort of 27,290 patients treated. The training cohort may covers seventy different cancer types with the 505 genes assessed by the IMPACT targeted sequencing oncology assay. The training ground truth labels may include oncogenic point mutations, copy number variations (amplifications or deletions) and fusion events, or the presence of these types of genetic variations in any of a group of genes canonically participating in a shared signaling pathway associated with cancer, e.g., DNA damage responses, RTK pathway, and mTOR signaling pathway as shown in Figure S2.

The task may be framed as a multi-label binary classification task, where genomic features (biomarkers) are represented as binary labels. Each binary label may indicate the presence or absence of genomic alterations in a single gene, or in any of a group genes that participate in a shared signaling pathway. The genomic feature binary labels derived from IMPACT results may be oncogenic mutations, copy number amplification, copy number deletions, fusions, or the combination of oncogenic mutation/amplification if a gene is an oncogene and oncogenic mutation/deletion if a gene is a tumor suppressor gene (TSG). Additional genomic features included in training may be TMB-H for TMB≥10 mutations/megabase (mut/Mb), MSI-H for MSI score ≥10, and microsatellite stable (MSS) for MSI score <3, dMMR for loss of IHC staining in MMR (MLH1, MSH2, MSH6, and PMS2) proteins and harbored genetic alterations in MMR genes, Lynch Syndrome for the presence of germline mutation in any of MLH1, MSH2, MSH6, PMS2, EPCAM, and CIN defined as presence of tetraploidy, whole genome doubling (WGD), loss-of-heterozygosity (LOH) in ≥50% genome, fraction of genome altered (FGA)≥30%, and genome instability index (GI index)≥0.2. The GI index may be a metric derived from FGA and LOH ranging from 0 to 1.

The development cohort may have been split into train, tune and test datasets with a ratio of 7:1:2. This partitioning may have resulted in sample sizes of 30,511 (33,564 WSIs) for the train set, 4,334 (4,762 WSIs) for the tune set, and 8,760 (9,634 WSIs) for the test set. The distribution of cancer types across the datasets may have included seventy different types in the train set, fifty-eight in the tune set, and sixty-two in the test set. Each dataset may include the category of an “Unknown” histology. A total of 1,228 biomarker labels with at least eight positive and eight negative samples in the train set and at least four positive and four negative samples in the tune set may have been included in the training.

Genomic features and/or binary biomarker labels may include 1) gene level alteration labels of 505 genes from MSK-IMPACT panel, 2) alterations in a group of genes participating in 5 canonical signaling pathways, 3) genome instability: tumor mutation burden (TMB), microsatellite instability high (MSI-H) or defects in mismatch repair genes (dMMR), and chromosomal instability (CIN) measured by fraction of genome altered (FGA), loss-of-heterozygosity (LOH), genome instability index (GI), whole genome doubling (WGD), and tetraploidy. The alterations may include mutations (SNVs/Indels), copy number aberration (amplification and deletion), and fusion events. The oncogenic status was determined based on OncoKB annotation.

Each slide may be split into image tiles of size 224×224 pixels. The tiles may be filtered to only include those representing foreground (tissue) using a foreground detection model based on a Fully Convolutional Network (FCN). Each foreground tile may be embedded with Virchow2 into a tile embedding of length 2560. Each slide may thus be represented as a N×2560 tensor, where N is the number of foreground tiles.

The embeddings may serve as input into a feed-forward network with an attention mechanism that aggregates the tile-level embeddings into a slide-level prediction. The model may have been trained on slide level. The final prediction per sample may have been determined as the maximum prediction over slides in the sample.

Checkpoint selection may have been done using the mean AUC and mean AP across all labels on the tune set. The operating threshold for each label may have been determined on the tune set by optimizing for 90% sensitivity in each cancer type present in the tune set. A threshold may correspond to a label and cancer type pair. These thresholds may then have been used to generate sample-level binary predictions from the inference probabilities in the test set and the TCGA validation set, indicating the presence or absence of the genetic mutation. From sixty-two cancer types present in the test set, fifty-six are also present in the tune set, thus we report results on the test set for these fifty-six cancer types.

### Phenotype-Genotype Correlation Analysis

For each histology subtype in a cancer, the Kolmogorov-Smirnov (KS) one-side test may have been implemented to examine whether the inference probabilities of a biomarker label in the target subtype is greater than the inference probabilities in the other subtypes of cancer. The p-values may have been adjusted using the Benjamini-Hochberg method. P-values <0.01 were considered as statistically significant. A histologic subtype ground truth may be defined as a binary label indicating if the sample was annotated for a given histologic subtype. For a biomarker with significantly higher inference probabilities in a specific histologic subtype of cancer, the AUC of the biomarker in prediction of the corresponding subtype may have been evaluated by using inference probabilities as prediction and the binary label of histologic subtype as ground truth. The sensitivity and specificity may have been computed by using the binary prediction of the genomic alterations as prediction label and the binary label of histologic subtype as ground truth.

### Financial Analysis

The cost-saving potential of using the model described herein may have been evaluated. The evaluation may be to triage for downstream molecular testing by excluding patients from downstream definitive NGS or PCR who are unlikely to harbor genetic mutations. It may be first assumed that this analysis is for a typical Phase 3 study seeking to enroll 500 patients with a mutation in a target gene in a particular type of cancer. BRAF (colorectal cancer and melanoma), EGFR (NSCLC), FGFR3 (bladder cancer), KRAS (NSCLC and colorectal cancer), and MET (NSCLC) may be selected as these are commonly assessed by NGS or PCR clinically, allowing good estimates of the cost of these molecular assays along with a nominal cost of AI model screening. Prevalence of these mutations in these forms of cancer may be assumed at published rates, with KRAS in NSCLC given a low estimate for a population with little to no smoking and a higher estimate for a population with high smoking prevalence. The estimated cost savings may be calculated due to reduced numbers of definitive NGS or PCR tests needed after the AI model identified cases unlikely to harbor the targeted mutation based on the performance of the AI model for these genes. The calculations may have been done according to the following method:

Assuming enrollment of a target number of patients all with tumors harboring a specific mutation, cost estimation for patient screening may have been calculated by 1) estimating cost to enroll the target number of patients using a conventional molecular test (NGS or PCR) only, and 2) NGS or PCR following pre-screening via an AI model to eliminate patients who were not likely to have the targeted mutation, hence would not be benefit from the targeted therapy or respond to targeted drugs. The cost savings may then have been calculated as follows:
Number of targeted enrolled patients: N_{target}
Prevalence of a genomic alteration in a cancer: prevalence
Sensitivity, i.e., the AI model algorithm's true positive rate: sensitivity
Specificity, i.e., the AI model algorithm's true negative rate: sensitivity
Cost of AI screening per patient: C_{AI}
Cost of molecular testing per patient: C_{testing}
Cost estimation for patient screening using a conventional molecular testing:
Number of patients to be screened by molecular testing without AI model:

$${N}_{conventional} = \frac{{N}_{target}}{prevalence}$$

Total cost of patients to be screened by molecular testing only without engaging an AI model:

$${C}_{conventional} = {C}_{testing} \times {N}_{conventional}$$

Cost estimation for patient screening using a molecular testing with an AI model for pre-screening:
Number of patients to be screened with an AI model:

$${N}_{screened} = \frac{{N}_{target}}{prevalence \times sensitivity}$$

Number of true positives (TP):

$$TP = {N}_{screened} \times prevalence \times sensitivity$$

Number of false positives (FP):

$$FP = {N}_{screened} \times ( 1 - prevalence ) \times ( 1 - specificity )$$

Number of patients sent for molecular testing:

$${N}_{sent} = TP + FP$$

Cost of AI model for screening all patients:

$${C}_{total ( AI )} = {C}_{AI} \times {N}_{screened}$$

Cost of molecular testing for patients sent for testing:

$${C}_{total ( testing )} = {C}_{testing} \times {N}_{sent}$$

Total cost of patients' molecular testing with AI screening:

$${C}_{with AI} = {C}_{total ( AI )} + {C}_{total ( testing )}$$

Total cost saving by using an AI model for pre-screening:

$${C}_{saving} = {C}_{conventional} - {C}_{with AI}$$

Hence, the percentage of cost reduction by using an AI model for prescreening:

$$C \% = \frac{{C}_{saving}}{{C}_{conventional}}$$

What is claimed is:

1. A computer-implemented method for processing at least one digital medical image to predict a first biomarker, the method comprising:
receiving the at least one digital medical image of one or more tissues of a patient, the at least one digital medical image including a plurality of tiles;
analyzing, via a foundation model, the plurality of tiles to determine an embedding vector for each of the plurality of tiles, the foundation model having been trained to predict embedding vectors at a tile-level based on a plurality of digital medical images; and
analyzing, via an aggregator model, the embedding vector for each of the plurality of tiles to predict the first biomarker of the digital medical image, wherein the aggregator model includes an attention mechanism configured to aggregate the embedding vector for each of the plurality of tiles into at least one slide-level prediction.

2. The method of claim 1, wherein the at least one digital medical image includes at least one of a whole slide image (WSI), a hematoxylin and eosin (H & E) stain, an immunohistochemistry (IHC) slide, an immunofluorescent slide, or a Computerized Topography (CT) scan.

3. The method of claim 1, wherein the biomarker is at least one of a genetic alteration biomarker, a histologic-subtype biomarker, a treatment-associated biomarker, a pathway biomarker, a chromosomal instability biomarker, a transcriptomic biomarker, a proteomic biomarker, an epigenetic biomarker, or a prognostic biomarker.

4. The method of claim 1, further including:
determining, based on the first biomarker, a diagnosis of a subtype of cancer.

5. The method of claim 1, wherein analyzing the plurality of tiles to determine the embedding vector for each of the plurality of tiles further includes:
analyzing, via a foreground detection model, the plurality of tiles to select a plurality of foreground tiles, the foreground detection model being a fully convolutional neural network trained to detect foreground in the plurality of tiles; and
analyzing, via the foundation model, the plurality of tiles to determine an embedding vector for each of the plurality of tiles, wherein the plurality of tiles comprises the plurality of foreground tiles.

6. The method of claim 1, further including:
determining, by the aggregator model, a second biomarker for the digital medical image, the second biomarker being a different biomarker type than the first biomarker.

7. The method of claim 1, further comprising:
analyzing, via a sizing model, the embedding vector for each of the plurality of tiles to predict a tumor size, the sizing model having been trained to predict the tumor size based on a plurality of embedding vectors.

8. The method of claim 1, further comprising:
analyzing, via a purity model, the embedding vector for each of the plurality of tiles to predict a tumor purity, the purity model having been trained to predict the tumor purity based on a plurality of embedding vectors.

9. The method of claim 1, further comprising:
based on the embedding vector for each of the plurality of tiles, generating a tile-level heatmap; and
generating a display including the tile-level heatmap overlaid on the at least one digital medical image.

10. The method of claim 9, further comprising:
based on the tile-level heatmap, generating a cell-level heatmap; and
generating the display including one or both of the tile-level heatmap or the cell-level heatmap overlaid on the at least one digital medical image.

11. The method of claim 10, wherein generating the cell-level heatmap is further based on the embedding vector for each of the plurality of tiles.

12. The method of claim 1, further comprising:
based on at least one of the first biomarker, a second biomarker, a tumor size, a tumor purity, a tile-level heatmap, or a cell-level heatmap, generating a request for review; and
transmitting the request for review to a third-party device.

13. The method of claim 1, wherein the aggregator model has been trained by:
receiving, as training data, a plurality of digital medical images associated with a plurality of patients and genomic abnormality data associated with the plurality of patients; and
training the aggregator model, using the training data, to infer the first biomarker of the digital medical image based on the respective one or more digital medical images.

14. The method of claim 13, wherein the training data further includes at least one of histological subtype data, treatment association data, genomic pathway data, or chromosomal instability data.

15. A method for training an aggregator model to predict at least one biomarker, comprising:
receiving a plurality of digital medical images associated with a plurality of patients;
receiving genomic abnormality data associated with the plurality of patients; and
training the aggregator model to predict the at least one biomarker based on the plurality of digital medical images and the genomic abnormality data.

16. The method of claim 15, wherein:
the at least one digital medical image includes at least one of a whole slide image (WSI), a hematoxylin and eosin (H & E) stain, an immunohistochemistry (IHC) slide, an immunofluorescent slide, or a Computerized Topography (CT) scan; and
the biomarker is at least one of a genetic alteration biomarker, a histologic-subtype biomarker, a treatment-associated biomarker, a pathway biomarker, a chromosomal instability biomarker, a transcriptomic biomarker, a proteomic biomarker, an epigenetic biomarker, or a prognostic biomarker.

17. A system for processing at least one digital medical image to predict a first biomarker, comprising:
at least one memory storing instructions; and
at least one processor configured to execute the instructions to perform operations comprising:
receiving the at least one digital medical image of one or more tissues of a patient, the at least one digital medical image including a plurality of tiles;
analyzing, via a foundation model, the plurality of tiles to determine an embedding vector for each of the plurality of tiles, the foundation model having been trained to predict embedding vectors at a tile-level based on a plurality of digital medical images; and
analyzing, via an aggregator model, the embedding vector for each of the plurality of tiles to predict the first biomarker of the digital medical image, wherein the aggregator model includes an attention mechanism configured to aggregate the embedding vector for each of the plurality of tiles into at least one slide-level prediction.

18. The system of claim 17, the operations further comprising:
analyzing, via a foreground detection model, the at least one digital medical image to generate the plurality of tiles, the foreground detection model being a fully convolutional neural network trained to detect foreground in the plurality of tiles,
wherein the plurality of tiles comprise tiles of the at least one digital medical image that include the foreground.

19. The system of claim 17, the operations further comprising:
at least one of:
determining, by the aggregator model, a second biomarker for the digital medical image, the second biomarker and the first biomarker being different,
analyzing, via a sizing model, the embedding vector for each of the plurality of tiles to predict a tumor size, the sizing model having been trained to predict the tumor size based on a plurality of embedding vectors,
analyzing, via a purity model, the embedding vector for each of the plurality of tiles to predict a tumor purity, the purity model having been trained to predict the tumor purity based on the plurality of embedding vectors, or
generating a tile-level heatmap and a cell-level heatmap based on the embedding vector for each of the plurality of tiles;
based on at least one of the first biomarker, the second biomarker, the tumor size, the tumor purity, the tile-level heatmap, or the cell-level heatmap, generating a request for review; and
transmitting the request for review to a third-party device.

20. The system of claim 17, wherein the aggregator model has been trained by:
receiving, as training data, a plurality of digital medical images associated with a plurality of patients and genomic abnormality data associated with the plurality of patients; and
training the aggregator model, using the training data, to infer the first biomarker of the digital medical image based on the respective one or more digital medical images.
